(19) United States
(12) Patent Application Publication
(10) Pub. No.: US 2023/0218173 A1
(43) Pub. Date: Jul. 13, 2023

(54) ENDOSCOPIC IMAGING AND PATTERNED STIMULATION AT CELLULAR RESOLUTION

(71) Applicant: Duke University, Durham, NC (US)

(72) Inventors: Henry Yin, Durham, NC (US); Jinyong Zhang, Durham, NC (US)

(21) Appl. No.: 18/080,868

(22) Filed: Dec. 14, 2022

Related U.S. Application Data
(60) Provisional application No. 63/289,394, filed on Dec. 14, 2021.

(51) Int. Cl. A61B 5/00; A61B 5/24

(52) U.S. Cl. CPC A61B 5/0071; A61B 5/24; A61B 18/20

(57) ABSTRACT

The present disclosure provides portable systems and methods of use thereof. In some aspects, provided herein are portable systems for in-vivo imaging. In some aspects, provided herein are portable systems for in-vivo two color calcium imaging. In some aspects, provided herein are portable systems for combined in-vivo imaging and optogenetics. In some aspects, provided herein are methods for combined modulation and imaging of cellular activity in vivo.

### STATEMENT REGARDING RELATED APPLICATIONS

This application claims priority to U.S. Provisional Patent Application No. 63/289,394, filed Dec. 14, 2021, the entire contents of which are incorporated herein by reference for all purposes.

### FIELD

The present disclosure provides portable systems and methods of use thereof. In some aspects, provided herein are portable systems for in-vivo imaging. In some aspects, provided herein are portable systems for in-vivo two color calcium imaging. In some aspects, provided herein are portable systems for combined in-vivo imaging and optogenetics.

### BACKGROUND

A major technological advance in neuroscience is the ability to record and manipulate neural activity with light. Readout of neural activity is made possible by the development of calcium indicators, such as genetically encoded calcium indicators, that bind to intracellular calcium and emit fluorescence that is proportional to neural activity. Manipulation of neural activity can be achieved by optogenetics, using different genetically expressed light-sensitive ion channels (opsins) to activate and inactivate specific neuronal populations. Together these techniques have revolutionized neuroscience, allowing investigators to record and manipulate neural activity with cell type specificity in behaving animals. Recently, these two tools have been combined to stimulate and record neural activity at the same time, but current approaches are large and unwieldy and are thus unsuitable for use in a freely-moving subject. Accordingly, what is needed are portable systems and methods for combined modulation and recording of cellular activity that can be used in freely moving animals, including freely moving rodents such as mice.

### SUMMARY

In some aspects, provided herein are portable systems for in-vivo imaging. In some embodiments, the portable system for in-vivo imaging comprises a single photon light source, a dichroic mirror, a freeform lens comprising an outer freeform surface and an inner plane, an implanted lens placed in a tissue of a subject; and an image sensor. In some embodiments, light rays from the single photon light source refract through the outer freeform surface onto the dichroic mirror, and are reflected by the dichroic mirror onto the implanted lens, thereby transferring light energy from the single photon light source to an illumination area on the tissue of the subject. In some embodiments, light energy transferred from the single photon light source to the tissue of the subject illuminates a detectable signal from a calcium indicator present in the tissue of the subject. In some embodiments, an image of the detectable signal is captured by the image sensor. In some embodiments, the outer freeform surface is designed such that light rays refracted through the outer freeform surface contact the dichroic mirror in locations that achieve a substantially even distribution of light rays relative to a center point within the illumination area following reflection. In some embodiments, the illumination area is a substantially circular area. In some embodiments, the illumination area has an average diameter of at least 150 microns. In some embodiments, the illumination area has an average diameter of about 250 microns.

In some embodiments, the portable system further comprises an excitation filter operably connected to the single photon light source to select for light of a first wavelength. In some embodiments, the portable system further comprises an emission filter. In some embodiments, the image sensor is a complementary metal oxide semiconductor (CMOS) image sensor. In some embodiments, the implanted lens is a gradient index (GRIN) lens. In some embodiments, the portable system further comprises an imaging lens, an electrotunable (ETL) lens, and an achromatic lens.

In some embodiments, the maximum distance between a point at the center of the inner plane and a point on the outer freeform surface is 1.5 mm or less. In some embodiments, the portable system weights 5 g or less. In some embodiments, the portable system weighs about 2.5 g. In some embodiments, the portable system is used in a method of in-vivo calcium imaging in a freely-moving mouse.

In some aspects, provided herein are portable systems for in-vivo two-color calcium imaging. In some embodiments, the portable system for in-vivo two color calcium imaging comprises a first light source and a second light source, an excitation dichroic mirror, an excitation lens, a main dichroic mirror, an implanted lens placed in a tissue of a subject, and a first image sensor and a second image sensor. In some embodiments, light rays from the first light source and the second light source are integrated into a main excitation path by reflection from the excitation dichroic mirror onto the excitation lens. In some embodiments, the light rays are subsequently refracted through the excitation lens onto the main dichroic mirror, and are then reflected by the main dichroic mirror onto the implanted lens, thereby transferring light energy from the first light source and the second light source to an illumination area on the tissue of the subject. In some embodiments, the excitation dichroic mirror is positioned such that the illumination area contains a substantial majority of the light rays from each of the first light source and the second light source. In some embodiments, the illumination area is a substantially circular area. In some embodiments, the illumination area has an average diameter of at least 150 microns. In some embodiments, the illumination area has an average diameter of about 250 microns. In some embodiments, first light source and the second light source are single photon light sources.

In some embodiments, the portable system further comprises a first light filter operably connected to the first light source to select for light of a first wavelength, and a second light filter operably connected to the second light source to select for light of a second wavelength.

In some embodiments, the light energy transferred to the tissue from the first light source illuminates a first detectable signal from a first calcium indicator present in the tissue of the subject, and the light energy transferred to the tissue from the second light source illuminates a second detectable signal from a second calcium indicator present in the tissue of the subject.

In some embodiments, the portable system further comprises an achromatic lens and an emission dichroic mirror. In some embodiments, the first detectable signal and the second detectable signal each refract through the achromatic lens onto the emission dichroic mirror, and are subsequently split by the emission dichroic mirror, thereby reflecting the first detectable signal to the first image sensor and the second detectable signal to the second image sensor.

In some embodiments, the implanted lens is a gradient index (GRIN) lens. In some embodiments, the first image sensor and the second image sensor are each complementary metal oxide semiconductor (CMOS) image sensors. In some embodiments, the excitation lens is a plane-convex lens, a drum lens, a half ball lens, or a freeform lens.

In some embodiments, the portable system weighs 5 g or less. In some embodiments, the portable system is used in a method of in-vivo two-color calcium imaging in a freely-moving mouse.

In some aspects, provided herein are portable systems for combined modulation and imaging of cellular activity in vivo. In some embodiments, the portable system for combined modulation and imaging of cellular activity in vivo comprises a laser, a collimation lens, and a spatial light modulator (SLM). In some embodiments, the portable system further comprises a single photon light source, an excitation dichroic mirror, an excitation lens, a main dichroic mirror, an implanted lens placed in a tissue of the subject, and an image sensor. In some embodiments, beams from the laser refract through the collimation lens to generate collimated laser beams. In some embodiments, the collimated laser beams are reflected by the spatial light modulator to generate a patterned excitation light path which is reflected by the main dichroic mirror onto the implanted lens, thereby transferring the patterned excitation light path to a stimulation area on the tissue of the subject and inducing patterned modulation of cellular activity within the stimulation area.

In some embodiments, patterned modulation of cellular activity comprises modulating activity of a single target cell within the stimulation area. In some embodiments, patterned modulation of cellular activity comprises modulating activity of a plurality of target cells within the stimulation area.

In some embodiments, the spatial light modulator comprises a plurality of operably independent mirrors.

In some embodiments, light rays from the single photon light source are reflected by the excitation dichroic mirror onto the excitation lens, refract through the excitation lens onto the main dichroic mirror, and are subsequently reflected by the main dichroic mirror onto the implanted lens, thereby transferring light energy from the single photon light source to an illumination area on the tissue of the subject. In some embodiments, the illumination area and the stimulation area are substantially the same region within the tissue. In some embodiments, the illumination area and the stimulation area are each substantially circular areas. In some embodiments, the illumination area and the stimulation area each have an average diameter of at least 150 microns. In some embodiments, the illumination area and the stimulation area each have an average diameter of about 250 microns.

In some embodiments, the light energy transferred from the single photon light source to the tissue of the subject illuminates a detectable signal from a calcium indicator present in the tissue of the subject, and an image of the detectable signal is generated by the image sensor.

In some embodiments, the portable system further comprises an excitation filter operably connected to the single photon light source to select for light of a first wavelength. In some embodiments, the portable system further comprises an emission filter. In some embodiments, the portable system further comprises an emission achromatic lens.

In some embodiments, the image sensor is a complementary metal oxide semiconductor (CMOS) image sensor. In some embodiments, the implanted lens is a gradient index (GRIN) lens. In some embodiments, the excitation lens is an achromatic lens.

In some embodiments, the portable system weights 8 g or less. In some embodiments, the portable system is used in a freely-moving mouse.

In some aspects, provided herein are methods for combined manipulation and imaging of cellular activity in vivo. In some embodiments, the method comprises connecting a portable system for combined modulation and imaging of cellular activity in vivo to a tissue of the subject. In some embodiments, the method further comprises generating beams from the laser, thereby transferring a patterned excitation light path to a stimulation area on the tissue and inducing a patterned manipulation of cellular activity for one or more cells within the stimulation area. In some embodiments, the method further comprises generating light rays from the one-photon light source, thereby illuminating a detectable signal from an indicator present in the tissue of the subject. In some embodiments, the method further comprises obtaining an image of the detectable signal. In some embodiments, the method further comprises converting the image of the detectable signal to a readout of a cellular activity pattern of one or more cells in the subject. In some embodiments, the method further comprises mimicking the cellular activity pattern of the one or more cells. In some embodiments, mimicking the cellular activity pattern of the one or more cells comprises selectively targeting the one or more cells with the patterned excitation light path, such that a patterned modulation of activity for the one or more cells is induced. In some embodiments, the indicator is a calcium indicator. In some embodiments, the one or more cells comprise neurons.

Other aspects and embodiments of the disclosure will be apparent in light of the following detailed description.

### BRIE DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic showing a network comprising an exemplary portable system for in-vivo imaging as described herein along with other components. The network is referred to as “Clear View”. As illustrated in FIG. 1, the network comprises a portable system for in-vivo imaging as described herein, referred to as the “Clear View Body” or “CVB”. The network further comprises a circuit board, referred to herein as the “Data Acquisition Board” or “DAB”, a behavior data recording interface (BDRI), and Data Recording Software (DRS). The network further comprises a computer.

FIG. 2 is a diagram of components for an exemplary system for in vivo imaging as described herein. This exemplary system, also referred to as the “Clear View Body”, comprises an excitation filter, emission filter, dichroic mirror, freeform lens, an electrotunable lens (ETL), an imaging lens (e.g. an achromatic lens), and GRIN lens, along with an image sensor (e.g. a CMOS).

FIG. 3 is a diagram of how exemplary components, such as those shown in FIG. 2, can be put together to form an exemplary system for in vivo imaging.

FIG. 4 is a schematic showing the principles of edge ray analysis that can be performed to generate a freeform lens for use in a portable system for in-vivo imaging as described herein.

FIGS. 5A-5D show a comparison of the illumination area and distribution for a system comprising a half-ball lens compared to a system comprising a freeform lens, as described herein. FIG. 5A shows the illumination area of the half-ball lens system, and FIG. 5B shows the illumination area of the freeform lens system. FIG. 5C is a schematic of an exemplary freeform lens design. FIG. 5D is a graph showing a comparison of energy distribution relative to a point on the center of the illumination area. The blue line shows the half-ball lens system, whereas the red line shows the energy distribution for a system described herein comprising a freeform lens.

FIG. 6 is a schematic of components of an exemplary image sensor for use in a portable system for in-vivo imaging described herein. In this embodiment, the image sensor is a CMOS. The CMOS comprises four sub-boards: a main CMOS board, a signal process board, an LED driver board, and an ETL driver board.

FIG. 7 is a schematic of components for an exemplary circuit board, referred to as a data acquisition board (DAB). The circuit board (e.g. DAB) is one component of a network comprising the portable system for in-vivo imaging as described herein. In this embodiment, there is only one circuit board, but it contains multiple functional areas including CMOS data flow collection, USB communication module, FPGA electronic control unit and behavior data acquisition unit. The FPGA electronic control unit processes multiple signals. It transmits the CMOS data flow to USB communication module immediately when it receives them from CMOS; and all the control signals from computer are decoded in this unit and sent to different ports. There is also a timer in the FPGA to synchronize the frame signals and the behavior data.

FIG. 8 is a drawing of an exemplary portable system for in-vivo imaging as described herein, also referred to as a “clear view body”. In this embodiment, the main CMOS board is fixed on the top of the body, and the signal process board is fixed on the side of the body. The signal process board covered with black epoxy resin. When it fixed on the side, it covers the excitation filter and dichroic mirror. The LED driver board is fixed on the narrow side of the body, and when it fixed, the freeform lens can be also fixed in front of it. The ETL is fixed at the bottom of the main body, and is connected with the ETL FPC. The entire Clear View Body weighs about 2.5 g. This tiny size and weight permits relatively small animals like mice to carry it during behavioral experiments without notable hinderance to their movement or natural behavior.

FIGS. 9A-B show an exemplary system for two-color calcium imaging as described herein. FIG. 9A shows an exemplary system with individual components labeled. The system (also referred to as an endoscope) comprises an excitation path and emission path. For the excitation path, two different wavelength LEDs are fixed on different location on the endoscope body. In this embodiment, one is blue LED (450 nm˜490 nm filter) for GCaMP calcium indicator excitation, and the other is lime LED (547 nm˜572 nm filter) for jRCaMP excitation. The light sources (e.g. the LEDs) are controlled by a circuit board, referred to herein as the data acquisition board (DAB). Two wavelengths of light are integrated in the main excitation path by reflection from the excitation dichroic mirror. For the emission path, the fluorescent signals from the two indicators are split by an emission dichroic mirror and radiate into different CMOS. An achromatic lens is used in the main emission path to make sure the imaging is clearly reached on the CMOS. FIG. 9B shows the system without the individual components labeled, in use on a rodent head (top), along with a front view and a side view of the system (bottom).

FIGS. 10A-10D show an exemplary configuration of the digital micromirror device (DMD) The DMD operates in two modes, on-state (FIG. 10A) and off-state (FIG. 10B). In the on-state, the micromirrors are tilted at α (+17°). This allows the collimated light to be reflected to the excitation path. In the off-state, the mirrors in the DMD are tilted at −α(−17°), which causes the collimated light to be reflected towards the absorber rather than to the excitation path. The whole DMD must be rotated by θ (13°) to create enough room for the collimated lens. FIG. 10C shows a model of the MAPSI pattern created in Tracepro, showing the pattern illumination produced by MAPSI. FIG. 10D shows a simulation result from FIG. 10C. Light scattering is less than 5 μm. Peak power for each square is higher than 2 mW, which is above the threshold for exciting neurons.

FIGS. 11A-11G show laser collimation and calibration. FIG. 11A (top) is a schematic drawing of laser light collimation. λ (20°) is radiation angle of the light from the fiber head. φ(100 μm) is diameter of laser head. λ is measured by the power meter. The focal distance of the collimation lens is 3.5 mm, and d is the diameter of the lens. FIG. 11A (bottom) shows simulation settings and test of collimation using the software Tracepro. FIG. 11B shows energy distribution of the MAPSI. The peak power radiated on the target screen is 3.8 mW/mm2, higher than what is needed to excite neurons. FIG. 11C shows energy distribution. FIG. 11D is a simulation showing collimation deviation as a function of fiber diameter. A lower deviation is desirable as it results in higher resolution. FIG. 11E shows that x-axis offset increases the offset of illumination. Ideally, the fiber head should be positioned at the focal point of the collimation lens. FIG. 11F shows that as the offset increases, the collimation deviation also increases, reducing the precision of the stimulation. FIG. 11G shows calibration of MAPSI. An image with 10 pixels diameter white spot and black background was projected on DMD. While moving the spot, the fluorescence signal was recorded and used for calibration.

FIG. 12 shows an exemplary design for MAPSI. Computer 1 is primarily responsible for collecting data from the camera and MAPSI. It also commands all the subsystems to send and receive data. The second computer is primarily responsible for the pattern control subsystem, which projects pattern images onto the DMD.

FIGS. 13A-13I show that the high resolution of the system for combined modulation and recording of cellular activity described herein (also referred to as “MAPSI”) allows patterned stimulation and calcium imaging. FIG. 13As (top) shows a schematic of the LED excitation path and the FOV (˜250 μm). FIG. 13A (bottom) shows a schematic of the patterned optogenetic excitation path. A checkered illumination pattern was used to demonstrate its ability to create patterns. FIG. 13B (left) shows a schematic of the setup to validate axial (z-axis) resolution. FIG. 13B (right) shows the resolution of stimulated beamlets on the x-y plane and x-z plane using the confocal microscope. FIG. 13C shows lateral (x and y) resolution. The FWHM of fluorescence (N=6 mice) is less than 10.5 μm on the x axis and 9.8 μm on they axis using a 10 μm diameter spot. FIG. 13D shows fluorescence intensity decreases as a function of laser spot offset. FIG. 13E shows axial (z-axis) resolution, left shows the light intensity along the z-axis using a 10 μm diameter laser beamlet (power=70 mW/mm2 507, n=6 stimulated neurons) and right shows the axial resolution (FWHM) of the photoactivation beam linearly increases with the spot diameter. FIG. 13F shows that there is a linear relationship between fluorescence diameter and the laser beam diameter (D). The minimum fluorescence diameter is 10 μm. FIG. 13G shows peak fluorescence of all recorded neurons as a function of distance from the stimulation location (0-500 ms after stimulation onset, 5 mice, neurons, power=70 mW/mm 2). FIG. 13H shows a comparison of signals from a SPN co-expressing both calcium indicator and opsin and a SPN expressing only the calcium indicator. FIG. 13I shows a representative stimulated neuron showing high fidelity responses to stimulation. All error bars indicate SEM.

FIGS. 14A-14D show a schematic and recordings from an exemplary system for combined modulation and recording of cellular activity as described herein, also referred to as “Miniscope with All-optical Patterned Stimulation and Imaging” (“MAPSI”). FIG. 14A is a drawing of MAPSI on a mouse head. jRCaMP1b, a red shifted calcium indicator, was used for imaging, and ChR2 for optogenetics. FIG. 14B is a schematic illustration of exemplary MAPSI components. Two light sources are in the excitation path: the lime LED excites jRCaMP1b and the blue laser light excites ChR2. Micromirrors on the DMD reflect a collimated laser beam, generating arbitrary stimulation patterns created and controlled by a computer. The focal plane of the excitation achromatic lens matches the GRIN lens focal plane to ensure high resolution. The emission path records calcium activity with a CMOS sensor. FIG. 14C is a histological image showing dSPNs (D1-cre mouse) that co-express jRCaMP1b and ChR2. FIG. 14D shows calcium transients showing excitation of stimulated neuron #1, but not of nearby neighboring neurons #2 and #3.

FIGS. 15A-15C show free movement using MAPSI and an exemplary stimulation pipeline. To reduce the weight of MAPSI carried by the mouse, a custom commutator was built. FIG. 15A shows the commutator attached to a 4 g pulley, and allows the wires to rotate without being tangled. FIG. 15B, top shows open field trajectory over 15 min of a representative mouse with 4 g UCLA Miniscope compared to the trajectory of the same mouse carrying the 7.8 g MAPSI. FIG. 15B, bottom shows total movement distance, peak speed, and average speed (N=7, 4 D1-cre mice and 3 A2A-cre mice, 15 minutes in the same open field platform). Unpaired t test analysis revealed no significant difference between mice carrying UCLA Miniscope and MAPSI in distance (p=0.1274) or peak speed (p=0.0826), but mice carrying MAPSI showed reduced average speed (p=0.0238). *p<0.05. FIG. 15C shows an exemplary stimulation pipeline: Simultaneously record and analyze the calcium activity as well as the behavior. Isolate the behaviorally relevant neurons. Find the neurons that co-express both ChR2 and jRCaMP1b. Target neurons with co-expression and replay activity.

FIGS. 16A-16D show resolution of MAPSI stimulation in freely behaving animals. FIG. 16A shows multiple neurons are selected for simultaneous stimulation. Five representative traces are shown on the right: top 3 are directly stimulated neurons, and bottom 2 are neighboring neurons that were not stimulated. FIG. 16B shows the calcium signal from dSPNs expressing jRCaMP1b and ChR2. When 5 selected neurons were stimulated, neighboring neurons were not activated. FIG. 16C shows the calcium signal from iSPNs co-expressing jRCaMP1b and ChR2 in a representative mouse. When stimulating 3 neurons, neighboring neurons were not excited by the stimulation. FIG. 16D shows a comparison of neuron 1 and neuron 6 from C, showing several representative trials. Neuron 1 is directly activated by photo-stimulation. Although the neighboring neuron 6 is often activated by the stimulation, the evoked activity is highly variable, with a long latency (˜500 ms). This pattern suggests that neuron 6 is indirectly activated, presumably via some circuit connection presumably involving multiple synapses.

FIGS. 17A-17G show stimulation of direct pathway neurons that are active during contraversive turning reproduces contraversive turning. FIG. 17A shows that 5 selected D1+dSPNs neurons from a representative mouse in an open field arena were stimulated. These neurons co-express ChR2 and jRCaMP1b. FIG. 17B shows behavioral data was aligned to start of contraversive turning (3 D1-cre mice, 6-22 trials per mouse). FIG. 17C shows all recorded dSPNs (3 D1-cre mice, 86 neurons) aligned to contraversive turning and sorted according to turning onset. FIG. 17D shows Calcium activity of 5 neurons in a representative mouse activated during contraversive turning onset. FIG. 17E shows the same 5 neurons were stimulated, while 5 neighboring neurons not related to turning were also stimulated. FIG. 17F shows stimulating 5 selected neurons produced more contraversive turning than stimulating unrelated neurons or stimulation in control mice (3 D1-cre mice, 5-25 trials of stimulated neurons, 4-20 trials per mouse; 2 control mice, 6-16 trials per mouse). FIG. 17G shows Stimulation of 5 turning related neurons significantly increased contraversive turning angle compared to controls and unrelated neurons stimulation (one-way ANOVA: Stim groups, F_{(2, 50)}=9.303 p=0.0004. Tukey's post hoc analysis revealed stimulating contraversive turning-related neurons resulted in more turning than stimulating neighboring neurons (p=0.0120) and controls (p=0.0007). Stimulating unrelated neurons did not produce more turning than controls (p=0.2819). All error bars indicate SEM.

FIGS. 18A-18G show selective stimulation of indirect pathway neurons that are active during ipsiversive turning recapitulates ipsiversive turning. FIG. 18A shows 4 selected A2A+ DLS neurons from a representative mouse in an open field arena were stimulated. Neurons co-expressing both ChR2 and jRCaMP1b are shown in the histological images. FIG. 18B shows behavioral data was aligned to the onset of ipsiversive turning relative to the hemisphere being recorded from (3 A2A-cre mice, 5-28 trials per mouse). FIG. 18C shows all recorded A2A+ neurons (3 A2A-cre mice, total of 354 neurons, >50 neurons per mouse) aligned to ipsiversive turning and sorted according to turning onset. FIG. 18D shows 4 neurons from a representative mouse were selected that were active during ipsiversive turning. Data was aligned to ipsiversive turning onset. FIG. 18E shows the same 4 neurons from one sample mouse were stimulated. Four neighboring neurons which not high related with turning were also stimulated. FIG. 18F shows optogenetic excitation of the 4 selected neurons produced significantly more ipsiversive turning compared to controls (no opsin expression) or unrelated neurons (3 A2A-cre mice, 6-30 trials per mouse for stimulated neurons, 3-16 trials per mouse for unrelated neurons; 2 control mice, 6-13 trials per mouse). FIG. 18G shows stimulation increased ipsiversive turning (one-way ANOVA: Stim, F_{(2,56)}=11, p<0.0001). Tukey's post hoc analysis revealed stimulation of turning-related neurons produced greater ipsiversive turning compared to stimulation of unrelated neurons (p=0.0096) or of controls (p<0.0001), but stimulating unrelated neurons did not produce more ipsiversive turning than controls (p=0.2932). All error bars indicate SEM.

FIGS. 19A-19C show contraversive turning behavior can be reliably elicited by stimulating as few as 3 dSPNs. FIG. 19A shows calcium imaging of the selected direct pathway neurons for photostimulation. Anywhere from one to five neurons were selectively stimulated. FIG. 19B shows optogenetically stimulating 3 or more neurons reliably produced contraversive turning. FIG. 19C shows turning was significantly higher when exciting 3 or more neurons (One-way ANOVA, F(2,174)=15.39, p=0.0006). Tukey's post hoc analysis revealed no significant difference between 2 neurons and 1 neuron (p=0.9997), no significant difference between 3 neurons and 1 neuron (p=0.3750), significant difference between 4 neurons and 1 neuron (p=0.0454), significant difference between 5 neurons and 1 neuron (p<0.05, **p<0.001, ****p<0.0001.

FIGS. 20A-20H show sequential and patterned stimulation of the direct pathway causes contraversive turning. FIG. 20A shows D1-Cre mice were used with MAPSI for single neuron sequence stimulation from a representative mouse. FIG. 20B shows five neurons were stimulated in two different sequences. The schematic shows the order and sequence of neurons that were stimulated. The two sequences either progressed laterally-to-medially (sequence #1) or medially-to-laterally (sequence #2) direction. FIG. 20C shows calcium traces for the selected neurons from each sequence, showing the time course of their activation. FIG. 20D and FIG. 20E show stimulation of the 5 neurons in both sequences greatly increased maximum contraversive turning compared to controls, but there was no difference of the direction of the sequence (one-way ANOVA, F_{(2,60)}=4.112, p<0.0001. Tukey's post hoc analysis revealed that both sequences produced significantly more contraversive turning than controls (p=<0.0001, p<0.0001), but were not different from each other (p=0.9277) (3 D1-Cre mice, 5-25 trials for #1, 4-20 trials for #2, 2 control mice, 6-18 trials per mouse). FIG. 20F shows a schematic of sweeping stimulation experiment where a 20 μm-wide bar moved horizontally, from lateral side to medial side (LM) or from medial side to lateral side (ML). FIG. 20G shows sweeping stimulation for 1 second increased contraversive turning compared to controls (left). There was a significant main effect of stimulation vs. non-stimulation (one-way ANOVA F_{(2,58)}=0.1825, p=0.8336. Tukey's post hoc analysis revealed that sequences LM vs ML was not significant: p=0.9996; but LM vs controls: p<0.0001, and ML vs. controls, p<0.0001, were significant; 3 D1-Cre, 4-17 trials LM, 6-25 trials for ML; 2 controls, 5-19 trials per mouse). FIG. 20H shows sweeping stimulation for 5 seconds significantly increased contraversive turning compared to controls. There was a main effect of stimulation (F_{(2,12)}=5.88, p=0.017). Tukey's post hoc analysis however revealed no significant difference between sequence #1 and #2 (p=0.74), but a significant difference between sequence #1 and controls (p=0.0023) and between sequence #2 and controls (p=0.049) (3 D1-Cre mice, LM: 5-21 trials per mouse, ML: 5-25 trials per mouse; 3 controls, LM and ML, 6-20 trials per mouse). All error bars indicate SEM.

(FIGS. 21A-21H demonstrate that sequential and patterned stimulation of the indirect pathway causes ipsiversive turning FIG. 21A shows A2A-Cre mice were used with MAPSI for single neuron sequence stimulation. FIG. 21B shows 4 neurons were optogenetically excited in two different sequences. The schematic shows the order and sequence of neurons that were stimulated. Rows indicate individual neurons and columns indicate individual time points. The two sequences either progressed laterally-to-medially (sequence #1) or medially-to-laterally (sequence #2). FIG. 21C shows calcium traces for the 4 selected neurons from each sequence, showing the time course of their activation. FIG. 21D and FIG. 21E show stimulation of the 4 neurons in both sequences significant increased ipsiversive turning compared to controls (one-way ANOVA, F_{(2,51)}=0.63, p=0.5367, #1 vs #2: p=0.6191, #1 vs control: p<0.0001, #2 vs control: p<0.0001, 3 A2A-cre mice, 3-19 trials of LM per mouse, 3-17 trials of ML per mouse; 2 controls, 6-18 trials per mouse). FIG. 21F is a schematic of sweeping stimulation experiment. A 20 μm-wide bar moved horizontally, from lateral side to medial side (LM) or from medial side to lateral side (ML). Also see Video S4. FIG. 21G shows sweeping stimulation for 1 second significant increased ipsiversive turning compared to controls. Sweeping from lateral to medial also produced more ipsiversive turning. Ipsiversive turning was significantly higher than controls for both sequences, however, were not significant compared to each other (one-way ANOVA, F_{(2,12)}=14.79, p=0.0006. Tukey's post hoc analysis reviewed that sequence #1 was not significantly different than sequence #2, p=0.24), but sequence 1 (p=0.0005 and sequence 2 (p=0.0092) were significantly greater than controls 3 A2A-cre, 4-12 trials of LM per mouse, 3-12 trials of ML per mouse, 2 controls, 4-12 trials per mouse). Schematic representation of sweeping photo-stimulation. FIG. 21H shows sweeping stimulation for 5 seconds significantly increased ipsiversive turning compared to controls, and the LM sweep produced more ipsiversive turning compared to the ML sweep. (one way ANOVA, F_{(2,12)}=47.68, p<0.0001, 3 A2A-cre mice, 3-10 trials per mouse for LM and 4-17 trials for ML; 2 control mice, 6-13 trials per mouse). Tukey's post hoc analysis reveals that both sequences produced more ipsiversive turning than control (p<0.0001 for both), LM vs ML: p=0.0132. All error bars indicate SEM.

### DETAILED DESCRIPTION

In some aspects, provided herein are portable systems and methods of use thereof for in-vivo imaging, including in-vivo calcium imaging. In some aspects, provided herein are portable systems and methods of use thereof for in-vivo two color calcium imaging. The systems described herein are lightweight and portable, and thus can be used in freely moving animals, including small rodent such as mice. Moreover, the portable systems for in-vivo imaging achieve a substantially even distribution of light rays relative to a center point within a relatively large illumination area, and are thus advantageous over existing systems.

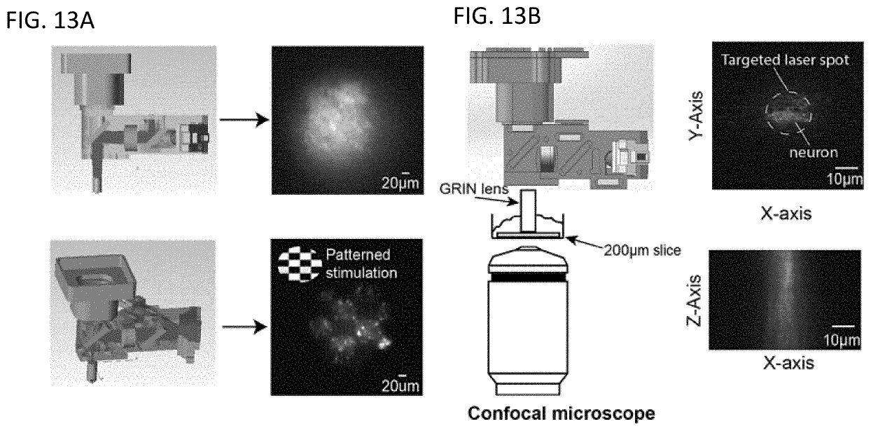

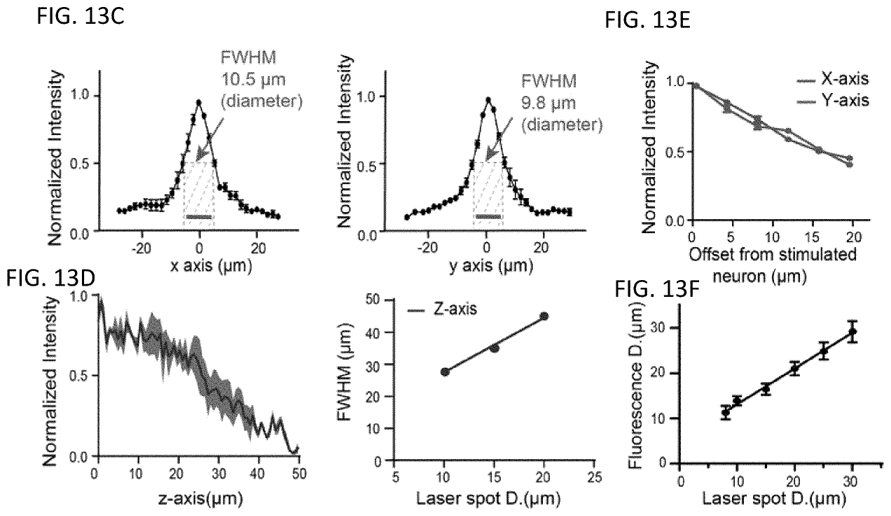

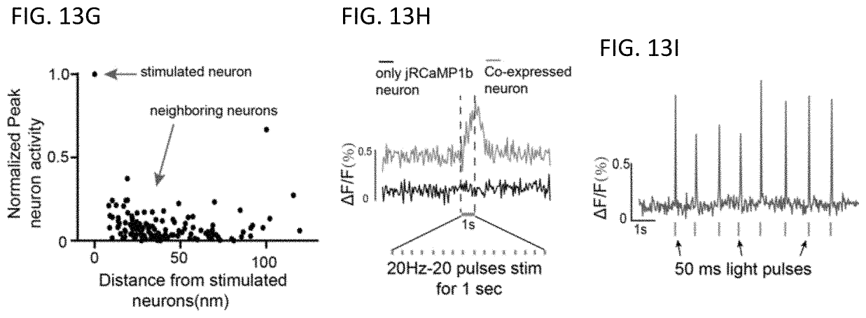

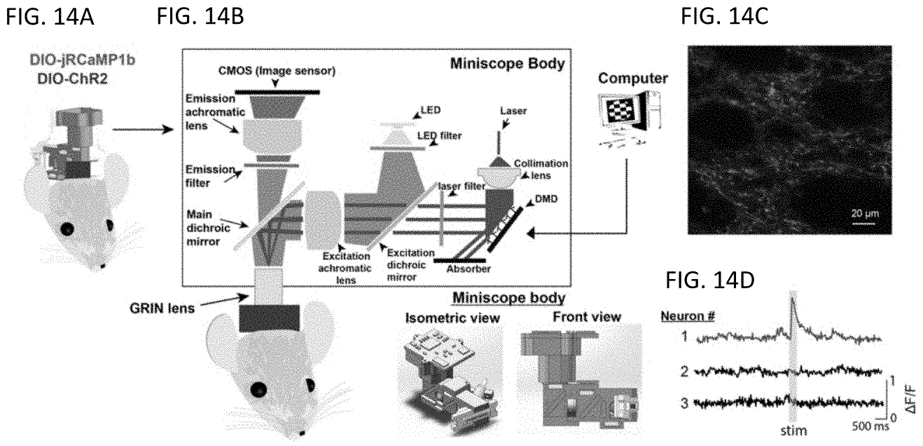

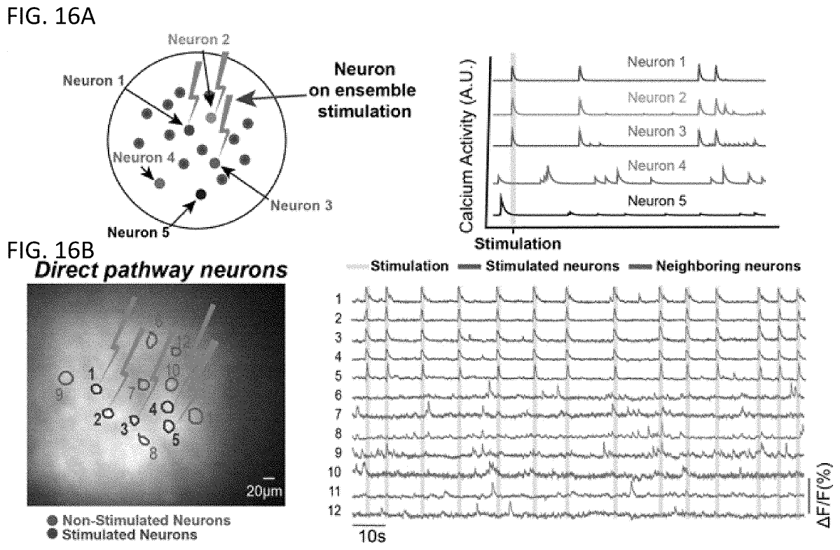

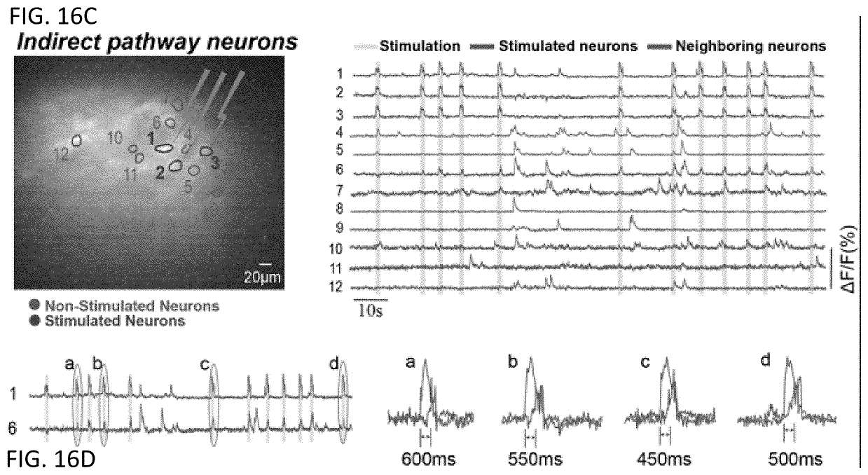

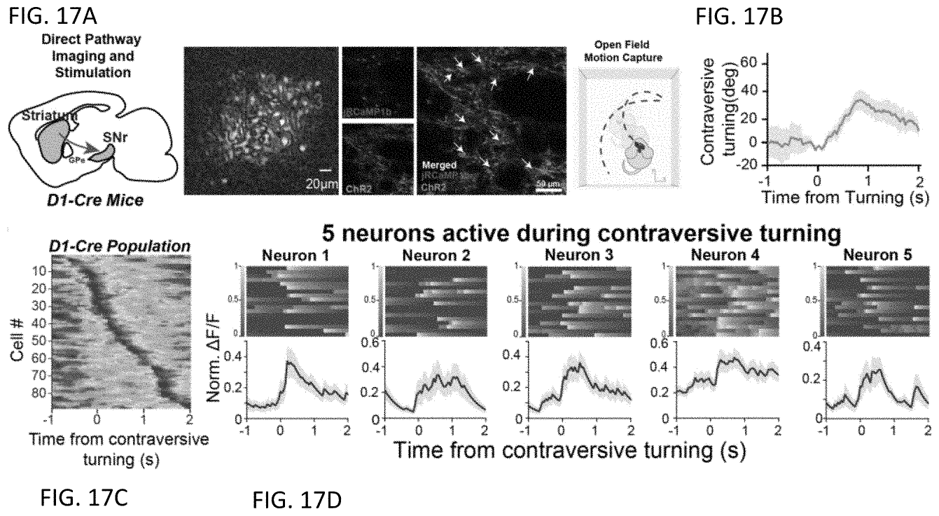

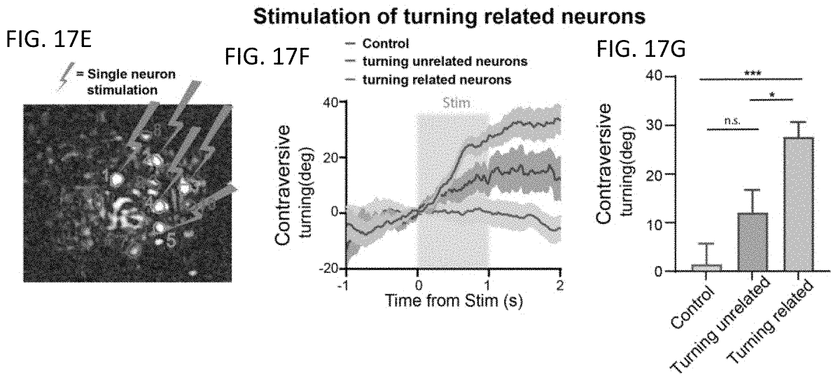

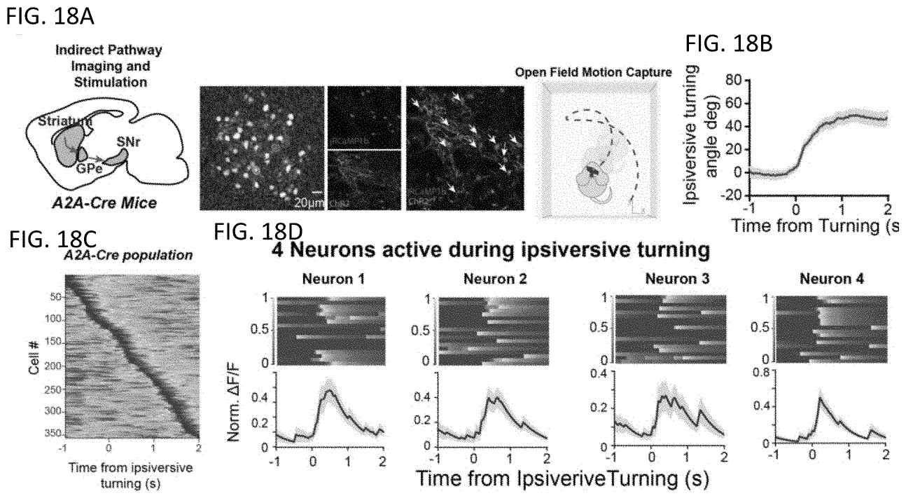

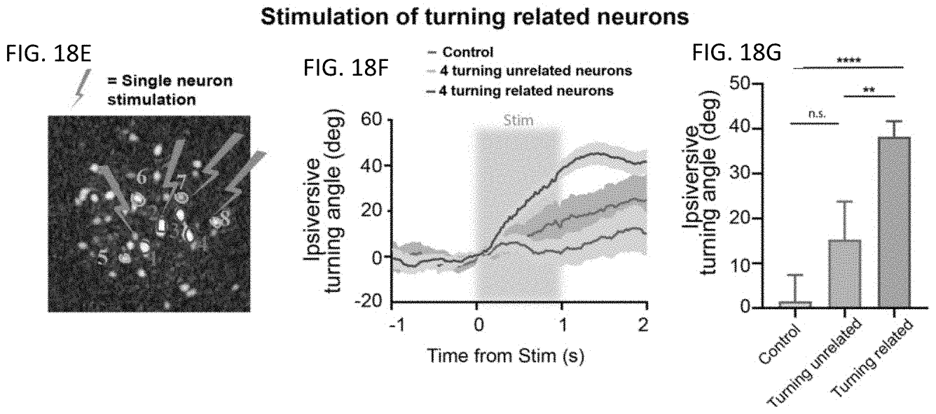

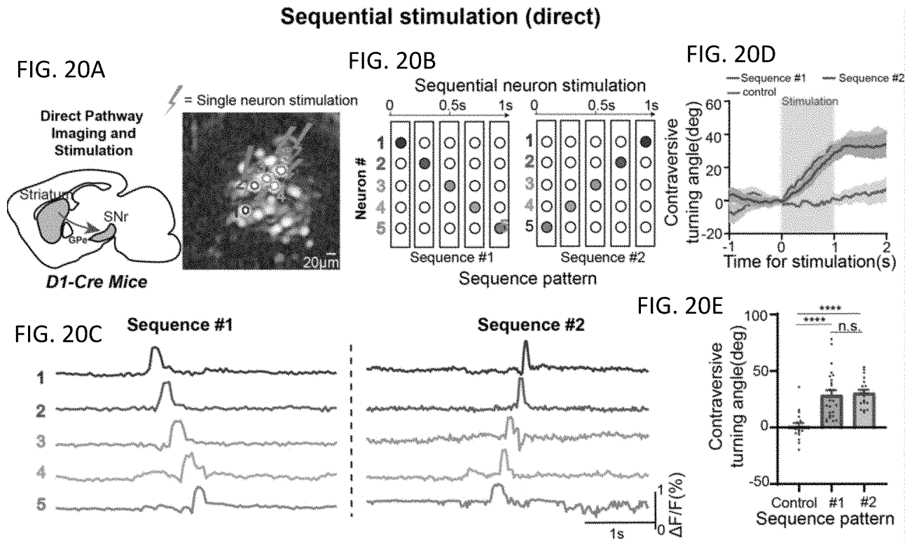

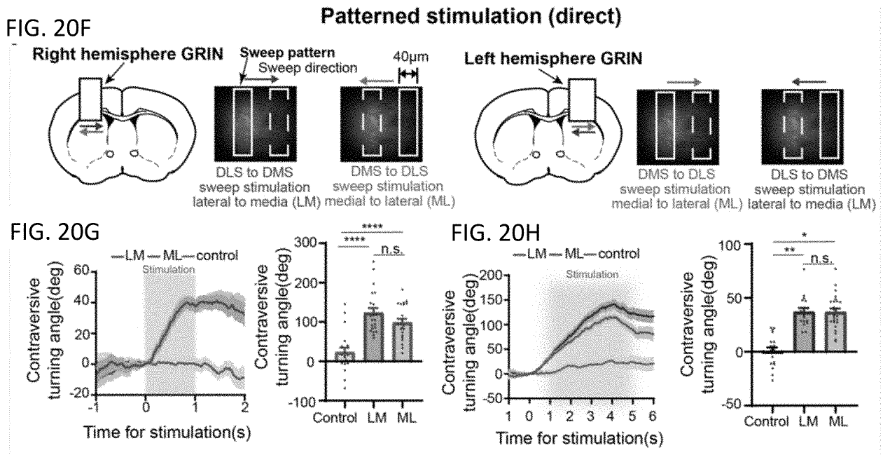

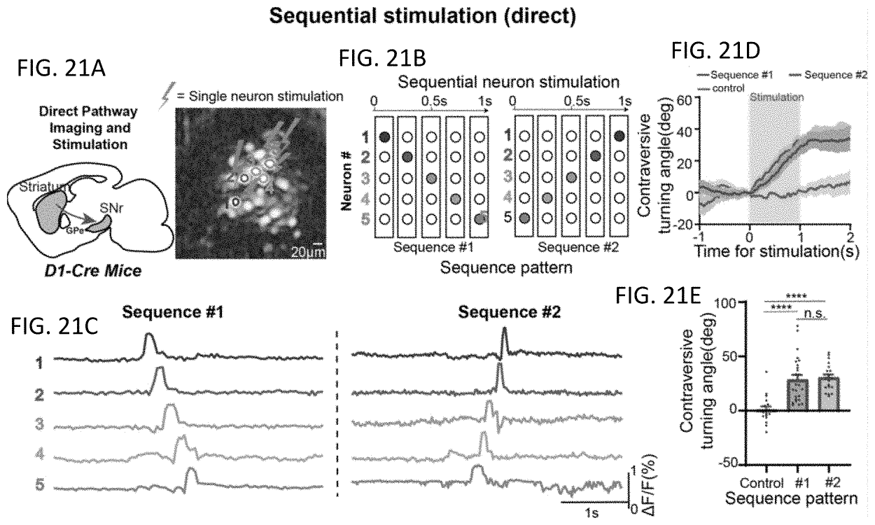

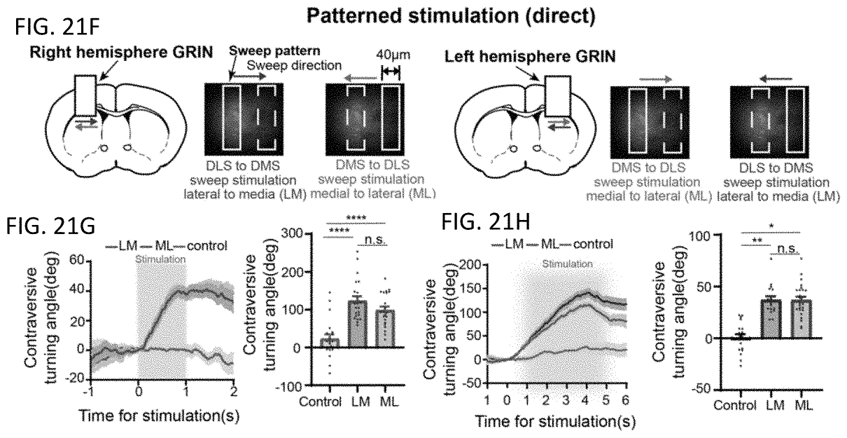

In some aspects, provided herein are portable systems for combined modulation and imaging of cellular activity in-vivo, and methods of use thereof. The system for combined modulation and imaging of cellular activity in vivo is also referred to herein as “MAPSI”. As described herein, the MAPSI system can: 1) provide near-cellular resolution stimulation (FIG. 13, FIG. 14), 2) stimulate and record from selected neurons and simultaneously record activity in other neurons in the FOV (FIG. 16), 3) identify neurons active during a specific behaviour and then selectively activate the relevant neuronal ensembles to reproduce the behaviour (FIG. 17, FIG. 18), 4) recreate the recorded spatiotemporal pattern with stimulation, and 5) synthesize arbitrary stimulation patterns in the FOV (FIG. 20, FIG. 21).

A major challenge in optogenetics is to control the spatial location and extent of photo-stimulation. Traditionally light delivery is achieved with flat-faced optical fibers which illuminate a relatively fixed brain volume around the tip of the fiber. Although recently developed optogenetic methods can control the extent of light delivery, it is difficult to generate patterned stimulation with high resolution in freely behaviour mice without using a two-photon (2P) setup. Although 2P methods can achieve cell-specific stimulation, they require expensive setups and often cannot be performed in freely moving animals. In contrast, MAPSI makes it possible to synthesize complex spatiotemporal sequences of neural activity in any brain region in freely moving animals.

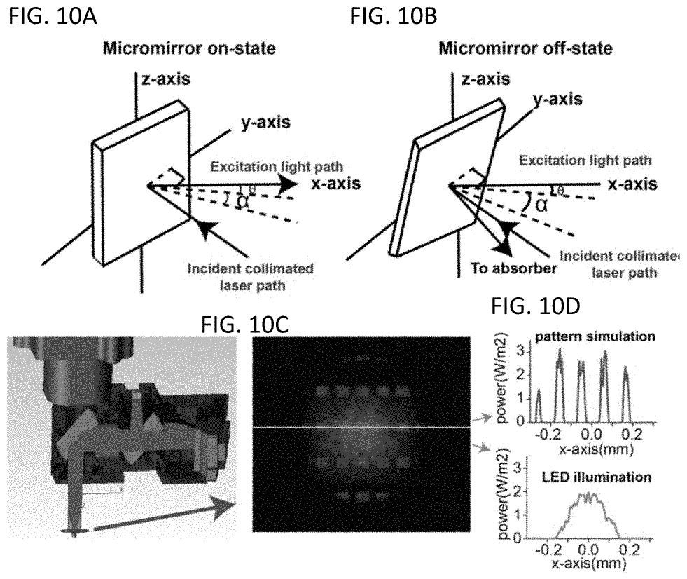

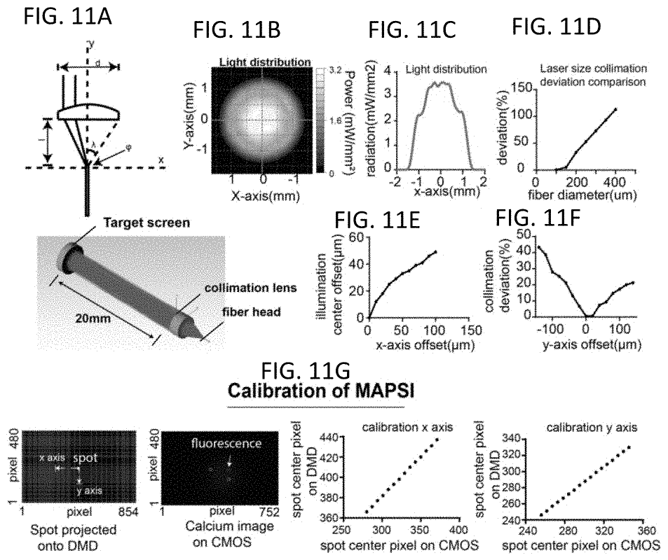

By using a spatial light modulator, such as a digital micromirror device (DMD), precise beamlets are achieved that can target single, or multiple, user-selected neurons with single-cell resolution. This design was tested in freely moving mice by simultaneously recording and stimulating direct and indirect pathway neurons in vivo. The recorded calcium activity was replayed in a small group of neurons to reproduce the same behaviour (FIG. 20, FIG. 21). This is the first demonstration that selective stimulation of a few SPNs could result in turning behaviors. In either direct or indirect pathways, activation of as few as 3 SPNs could produce significant turning, contraversive for dSPNs and ipsiversive for iSPNs. The amount of turning depends on the number of turning-related neurons activated, and activation of 5 neurons produced a comparable amount of turning as stimulation of the entire field of view (FOV) (FIG. 10, FIG. 11). In the field of view, the number of SPNs that are specifically related to turning is low, and stimulating the whole field would additionally activate neurons with presumably other functions. These results suggest that the number of SPNs involved in generating a specific action is much lower than expected.

MAPSI represents a major advance over existing systems for simultaneous imaging and photo-stimulation. Current systems that are capable of patterned stimulation and imaging usually require large microscopes and are difficult to use in freely-moving animals. Examples of existing 1P system is a fiberscope system developed by Szabo and colleagues (Szabo, V., et al., Neuron 84, 1157-1169 (2014)) and a commercially available 1P fiberscope (OASIS Implant). However, each of these systems have several limitations. Namely, these systems require a confocal microscope as well as two separate lasers, whereas the system provided herein does not require a separate microscope and uses only one laser. Accordingly, the system herein is more lightweight and portable, rendering it significantly more useful for in-vivo experiments in a relatively small subject such as a mouse. Secondly, with existing systems, lateral and axial resolution degrade when the animal is moving with the optic fiber. Moreover, the wavelength for ChR2 stimulation overlaps with the excitation wavelength of the calcium indicator (GCaMP5), so some neurons could be excited during calcium imaging. The FOV for stimulation and recording in existing systems is also limited by the properties of the coupled micro-objective. In contrast, the system herein uses a GRIN lens, which makes it possible to move the baseplate to move the FOV to a different region covered by the lens. Thus many neurons can be recorded and stimulated from a single animal.

MAPSI, because the dual-beam paths are independent of each other, neurons selected for stimulation can be simultaneously recorded along with the activity of other neurons in the FOV. It is therefore the first system that allows both manipulation and recording of any recorded neuron at the same time in freely moving animals. As such, MAPSI can be used to interrogate neuronal circuit function and in investigating mouse models of neurological and psychiatric disorders. In short, given its low cost and portability, MAPSI provides a powerful new tool for understanding the neural basis of behavior.

Section headings as used in this section and the entire disclosure herein are merely for organizational purposes and are not intended to be limiting.

### 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “calcium indicator” is used herein in the broadest sense and refers to a molecule that produces a detectable signal in response to binding of calcium ion(s) to the indicator. Generally speaking, calcium indicators contain moieties that produce a detectable signal in response to binding of calcium ion(s) to the indicator. Some calcium indicators contain fluorescent moieties that produce a fluorescent signal in response to binding of calcium ion(s) to the indicator. Some calcium indicators contain luminescent moieties that produce a luminescent signal in response to binding of calcium ion(s) to the indicator. The term “calcium indicator” is inclusive of both “chemical indicators” and “genetically encoded calcium indicators” or “GECI”.

The term “chemical indicator” when used in reference to a calcium indicator refers to a small molecule indicator that chelates calcium ions. Chemical indicators are based on a calcium-specific aminopolycarboxylic acid. Some calcium indicators are based on the calcium-specific aminopolycarboxylic acid (1,2-bis(o-aminophenoxy)ethane-N,N,N′,N′-tetraacetic acid) (BAPTA), which possesses high selectivity for calcium ions compared to magnesium ions. Chemical indicators are typically lipophilic to permit easy entry into the cell, where the indicator binds to calcium if present in the cell. Binding of calcium produces a detectable signal, such as an increase in quantum yield of fluorescence, or a shift in the emitted wavelength of the indicator. Exemplary chemical indicators include, for example, fura-2, indo-1, fluo-3, fluo-4, and Calcium Green-1.

The term “genetically encoded calcium indicator” or “GECI” are used interchangeably herein in the broadest sense to refer to an indicator wherein a gene encoding at least one reporter protein is transfected into cells, and subsequent binding of calcium to a moiety within the indicator results in a detectable signal. The at least one reporter protein may be a fluorescent or luminescent protein. Broadly speaking, GECIs comprise a calcium sensor which binds to calcium, and at least one reporter protein which produces a detectable signal upon binding of calcium to the sensor. A common sensor is calmodulin (CaM), however other suitable calcium sensors may be used including troponin C and derivatives thereof (e.g. modified chicken skeletal muscle troponin C, human cardiac troponin C). Common reporter proteins include fluorescent reporter proteins such as green fluorescent protein (GFP) or others (e.g. GFP, BFP, YFP, CFP, RFP, etc.) and bioluminescent reporter proteins such as luciferin, aequorin, obelin, and derivatives thereof. The term “GECI” is inclusive of single protein systems and paired protein systems. Whereas single protein systems rely on a single reporter protein (e.g. GFP, YFP, CFP, etc.) to produce a detectable signal, paired protein systems rely on transfer of energy from a first protein to a second protein for the signal to be detected. For example, paired fluorescent protein systems may utilize a forster resonance energy transfer (FRET) pair, which may be any suitable pair of fluorescent proteins.

The term “dichroic mirror” refers to a mirror with different reflection or transmission properties at two different wavelengths.

The term “freely-moving” as used herein refers to a subject being able to move and engage in natural behaviors without substantial hinderance.

The term “gradient index lens” or a “GRIN lens” refers to a lens that produces a gradient of refraction of light, thus reducing aberrations and permitting multiple beams of light to converge upon a single focal point.

The term “modulating” or “modulation” when used in reference to cellular activity is used in the broadest sense and includes both increasing cellular activity (e.g. enhancing or stimulating cellular activity) or diminishing cellular activity (e.g. inhibiting cellular activity).

The term “nervous tissue” or “neural tissue” are used interchangeably herein and refer to a tissue of the nervous system, including the central nervous system and the peripheral nervous system.

The term “single photon light source” refers to a light source that emits light as a single particle (i.e. a single photon). A single photon light source is in contrast to a laser, a multi-photon light source, or a thermal light source such as an incandescent light bult.

The term “portable” as used herein refers to a system that can be connected to a subject and thereby move with the subject. A “portable” system is in contrast to a “stationary” system, which may be tethered to a subject (e.g. by a rope, a cord, or the like) thereby permitting movement of the subject, but does not move with the subject. The portable systems described herein can be lightweight to facilitate use in a freely moving subject, without substantially hindering the movement of the subject.

The term “rodent” refers a mammal of the order Rodentia, including but not limited to mice, rats, guinea pigs, and hamsters.

### 2. Portable Systems and Methods for In-Vivo Imaging

In some aspects, provided herein are portable systems and methods of use thereof for in-vivo imaging. In some embodiments, provided herein is a portable system for in-vivo imaging. The portable system for in-vivo imaging is also referred to herein as a portable system for “one-photon imaging”. In some embodiments, the portable system comprises a single-photon light source (also referred to as a one-photon light source), a dichroic mirror, a freeform lens, an implanted lens, and an image sensor. In some embodiments, light rays from the single photon light source refract through the freeform lens onto the dichroic mirror, and are reflected by the dichroic mirror onto the implanted lens, thereby transferring light energy from the single photon light source to an illumination area on the tissue of the subject. The illumination area is also referred to herein as the “field of view” or “FOV”.

In some embodiments, the illumination area is a substantially circular area. In some embodiments, the illumination area has an average diameter of at least 150 microns. In some embodiments, the illumination area has an average diameter of at least 150 microns, at least 160 microns, at least 170 microns, at least 180 microns, at least 190 microns, at least 200 microns, at least 210 microns, at least 220 microns, at least 230 microns, at least 240 microns, or at least 250 microns. In some embodiments, the illumination area has an average diameter of about 250 microns.

In some embodiments, the light energy transferred from the single photon light source to the tissue of the subject illuminates a detectable signal from an indicator present in the tissue of the subject. An image of the detectable signal can then be generated by the image sensor. In some embodiments, the indicator is a calcium indicator. The indicator may be any suitable calcium indicator, including a chemical indicator or a genetically encoded calcium indicator (GECI). In some embodiments, the portable system is used for in-vivo calcium imaging. In some embodiments the light energy transferred from the single photon light source to the tissue of the subject illuminates a detectable signal from a calcium indicator present in the tissue of the subject, and an image of the detectable signal is captured by the image sensor.

In some embodiments, the system comprises a single photon light source. Use of a single-photon light source may assist in ultimately creating a system that is lightweight and portable, such as for use in in-vivo imaging (e.g. in-vivo calcium imaging) in a relatively lightweight subject, such as a mouse or a rat. In some embodiments, the system comprises single photon light source and a dichroic mirror.

In some embodiments, the system additionally comprises a freeform lens comprising an outer freeform surface and an inner plane. The term “freeform” indicates that the outer surface of the lens has been generated to ultimately optimize energy distribution (e.g. distribution of light rays) on the illumination area. In some embodiments, the outer freeform surface is designed such that light rays refracted through the outer freeform surface contact the dichroic mirror in locations that achieve a substantially even distribution of light rays relative to a center point within the illumination area following reflection. For example, distribution of light rays on the dichroic mirror can be analyzed and used to generate the outer freeform surface of the freeform lens such that the light rays ultimately contact the dichroic mirror at optimized locations, thereby reflecting off of the dichroic mirror and onto the illumination area with a substantially even distribution. An exemplary method for optimizing the freeform lens (e.g. designing the freeform surface) is described in Example 1.

The systems described herein are advantageous over other existing calcium imaging systems, at least in part due to the substantially even distribution of light rays achieved within the illumination area, thereby providing a relatively large illumination area (e.g. at least 150 microns) while avoiding photo bleaching of cells in the center of the illumination area. For example, the systems described herein comprising a freeform lens optimized to achieve the desired distribution of light rays on the dichroic mirror permit visualization of a relatively large illumination area on the tissue, such as a roughly circular illumination area having an average diameter of of at least 150 microns, at least 160 microns, at least 170 microns, at least 180 microns, at least 190 microns, at least 200 microns, at least 210 microns, at least 220 microns, at least 230 microns, at least 240 microns, or at least 250, wherein the distribution of light rays within the illumination area is substantially even. Moreover, the systems are lightweight and portable, and are thus advantageous for use in a freely moving subject.

In some embodiments, the maximum distance between a point at the center of the inner plane of the freeform lens and a point on the outer freeform surface is 1.5 mm or less. For example, in some embodiments, the maximum distance between a point at the center of the inner plane of the freeform lens and a point on the outer freeform surface is 1.5 mm, 1.4 mm, 1.3 mm, 1.2 mm, 1.1 mm, 1.0 mm, 0.9 mm, 0.8 mm, 0.7 mm, 0.6 mm, or 0.5 mm.

In some embodiments, the system further comprises an implanted lens placed in the tissue of a subject. In some embodiments, the implanted lens is a gradient index (GRIN) lens. In some embodiments, the GRIN lens comprises a flat lens surface. In some embodiments, the GRIN lens comprises a curved lens surface. In some embodiments, the GRIN lens is cylindrical.

The implanted lens (e.g. the GRIN lens) may be placed in any desired tissue of the subject to facilitate in-vivo imaging within the tissue. In some embodiments, the implanted lens is placed in a nervous tissue of the subject. In some embodiments, the implanted lens is implanted in a central nervous system tissue. In some embodiments, the implanted lens is implanted in the brain of the subject. In some embodiments, the planted lens is implanted in the spinal cord of the subject. In some embodiments, the implanted lens is implanted in peripheral nervous tissue, such as a peripheral nerve of the subject. In some embodiments, the system comprises a GRIN lens implanted in the brain of the subject. The location of the implanted lens may be modified, such as in between experiments, to permit imaging in multiple locations within the same subject.

In some embodiments, the system further comprises an image sensor. The image sensor assists in producing an image of the detectable signal that is illuminated upon light energy reaching the illumination area within the tissue. In some embodiments, the image sensor converts light waves into information that is used to make an image. In some embodiments, the image sensor converts light waves into bursts of current, which convey the information used to make the image. In some embodiments, the image sensor is a complementary metal oxide semiconductor (CMOS) image sensor.

In some embodiments, the system further comprises an excitation filter operably connected to the single photon light source to select for light of a first wavelength. For example, the excitation filter may filter out light of undesired wavelengths, thus selecting for light of the first wavelength (e.g. a desired wavelength). The first wavelength may be any desired wavelength and may depend on the indicator present within the tissue of the subject. Different calcium indicators are illuminated in the presence of light of different wavelengths. Accordingly, the excitation filter may select for light of a first wavelength that illuminates a signal from a calcium indicator present in the tissue of the subject. For example, if the calcium indicator present in the tissue is GCaMP6s, the excitation filter should select for blue light, such as blue light having a wavelength of 450 nm-490 nm, to permit visualization of the detectable signal from the GCAMP6s upon binding of calcium. As another example, if the calcium indicator is jRCaMP1b, the excitation filter should select for orange light, such as orange light having a wavelength of 550 nm-590 nm, to permit visualization of the detectable signal from the jRCaMP1b upon binding of calcium. These exemplary calcium indicators and filters are in no way to be construed as limiting to the calcium indicators that may be used in conjunction with the system described herein or the filters to be used to visualize said indicators. Any suitable first wavelength or filter for selecting the first wavelength may be used depending on the given calcium indicator present in the tissue of the subject.

In some embodiments, the system further comprises an emission filter. In some embodiments, the emission filter constrains the emission light wavelength, thereby preventing disturbing light that may otherwise interfere with imaging.

In some embodiments, the system further comprises one or more additional lenses to facilitate generation of a clear image on the image sensor. For example, the system may further comprise one or more additional lenses selected from one or more imaging lenses, an electrotunable (ETL) lens, and an achromatic lens. In some embodiments, the system comprises an imaging lens. The imaging lens can be a single lens or a set of lenses that work in conjunction. The imaging lens can be used to change the working distance for generating an image on the image sensor. The imaging lens can be used to match the focal plane of the GRINS lens. Adjusting the distance from the imaging lens to the GRIN lens can alter the quality of the image, and thus the imaging lens can be used to generate a clear image on the image sensor. The ETL lens can be used to adjust the focal plane of the imaging lens, for example to enhance the working distance by electrical modulation. For example, if the imaging lens working distance is only 0.2 mm, the ETL can increase the working distance to 0.5 mm-1.5 mm. The achromatic lens removes parallax error or perspective error. The achromatic lens can also magnify the imaging path and and influences the size of the field of view. In some embodiments, the system further comprises an imaging lens, an ETL lens, and an achromatic lens.

In some embodiments, the system weighs about 5 g or less. In some embodiments, the system weighs about 4 g or less. In some embodiments, the system weighs about 3 g or less. In some embodiments, the system weighs about 5 g, about 4.9 g, about 4.8 g, about 4.7 g, about 4.6 g, about 4.5 g, about 4.4 g, about 4.3 g, about 4.2 g, about 4.1 g, about 4.0 g, about 3.9 g, about 3.8 g, about 3.7 g, about 3.6 g, about 3.5 g, about 3.4 g, about 3.3 g, about 3.2 g, about 3.1 g, about 3.0 g, about 2.9 g, about 2.8 g, about 2.7 g, about 2.6 g, about 2.5 g, about 2.4 g, about 2.3 g, about 2.2 g, about 2.1 g, or about 2.0 g. In some embodiments, the system weights about 2.5 g.

In some embodiments, the portable system is incorporated into a network. In some embodiments, the network comprises the portable system, along with one or more additional components that control one or more components of the portable system (e.g. control one or more lenses, control the single-photon light source, control the image sensor, etc.) and/or operate in conjunction with the portable system. For example, in some embodiments the network comprises the portable system, along with a computer. In some embodiments, the network comprises the system, along with a circuit board. In some embodiments, the network comprises the portable system, a computer, and a circuit board. In some embodiments, the circuit board loads data (e.g. calcium imaging data, behavioral recording data, data regarding the state of the portable system itself) to the computer. In some embodiments, the circuit board is operably connected to one or more components of the portable system (e.g. one or more mirrors, lenses, the light source, etc.). In some embodiments, the circuit board is operably connected to the one or more components of the portable system in that it transmits signals to the one or more components of the portable system. For example, in some embodiments the circuit board transmits signals to the light source, thereby directing the operation thereof. In some embodiments, the circuit board transmits signals to one or more lenses, thereby directing the operation thereof. In some embodiments, the network comprises a behavioral data recording interface (BRDI). In some embodiments, the behavioral data recording interface comprises a camera, which records behaviors of the subject. In some embodiments, the BRDI comprises a camera, which records behavior of the subject while connected to the portable system (e.g. while performing calcium imaging on the subject). In some embodiments, the BRDI is operably connected to the circuit board, such that the circuit board can transmit signals to the BRDI to control the function thereof. In some embodiments, the circuit board additionally transmits data from the BRDI to the computer. In some embodiments, the computer executes a software program, such as a software program containing instructions to perform a given calcium-imaging method.

In some embodiments, the system or network comprising the same is used in a method of in-vivo imaging in a freely-moving subject. In some embodiments, the subject is a rodent. In some embodiments, the subject is a mouse. In some embodiments, the system or network comprising the same is used in a method of in-vivo calcium imaging in a freely moving mouse. In some embodiments, the method of in-vivo imaging in a freely-moving subject comprises connecting the portable system to the subject, generating light rays from the single-photon light source, and collecting data from the subject. In some embodiments, the method comprises connecting the portable system to the subject, generating light rays from the single-photon light source (e.g. by executing a software program on the computer, which directs the circuit board to control the single-photon light source), thereby transferring light energy from the single-photon light source to the illumination area of the subject. In some embodiments, the light energy transferred from the single photon light source to the tissue of the subject illuminates a detectable signal from a calcium indicator present in the tissue of the subject, and the method further comprises capturing an image of the detectable signal. In some embodiments, the cells of the subject (e.g. cells within the illumination area) express or contain a calcium indicator. In some embodiments, the cells express a genetically encoded calcium indicator, such that the light rays generated from the one-photon light source illuminates a detectable signal from the indicator present in the tissue in the subject.

### 3. Portable Systems and Methods for In-Vivo Two Color Calcium Imaging

In some embodiments, provided herein are portable systems for in-vivo two color calcium imaging. In some embodiments, the system comprises a first light source and a second light source. The first light source and the second light source are separate and distinct light sources. In some embodiments, light rays from the first light source and the second light source are integrated into a main excitation path, such that light energy from the first light source and the second light source are transferred to an illumination area on the tissue of the subject.

In some embodiments, the system comprises a first light source and a second light source, an excitation dichroic mirror, an excitation lens, a main dichroic mirror, an implanted lens, and a first image sensor and a second image sensor. In some embodiments, light rays from the first light source and the second light source are integrated into a main excitation path by reflection from the excitation dichroic mirror onto the excitation lens, are subsequently refracted through the excitation lens onto the main dichroic mirror, and are then reflected by the main dichroic mirror onto the implanted lens, thereby transferring light energy from the first light source and the second light source to an illumination area on the tissue of the subject.

In some embodiments, the excitation dichroic mirror is positioned such that the illumination area contains a substantial majority of the light rays from each of the first light source and the second light source. The term “substantial majority” indicates that the illumination area contains at least 80% of the light rays from each of the first light source and the second light source. For example, in some embodiments a “substantial majority” indicates that the illumination area contains at least 80%, at least 85%, at least 90%, at least 91%, at least 92%, at least 93%, at least 94%, at least 95%, at least 96%, at least 97%, at least 98%, or at least 99% or the light rays from each of the first light source and the second light source.

In some embodiments, the illumination area is a substantially circular area. In some embodiments, the illumination area has an average diameter of at least 150 microns. In some embodiments, the illumination area has an average diameter of at least 150 microns. In some embodiments, the illumination area has an average diameter of at least 150 microns, at least 160 microns, at least 170 microns, at least 180 microns, at least 190 microns, at least 200 microns, at least 210 microns, at least 220 microns, at least 230 microns, at least 240 microns, or at least 250 microns. In some embodiments, the illumination area has an average diameter of about 250 microns.

In some embodiments, the position of the excitation dichroic mirror is sufficient to ensure that the illumination area contains a substantial majority of the light rays from each of the first light source and the second light source, without requiring additional lenses to eliminate aberrations. For example, in some embodiments the position of the excitation dichroic mirror is sufficient to ensure that the illumination area contains a substantial majority of the light rays from each of the first light source and the second light source, without using an achromatic lens between the first and second light source and the main dichroic mirror to reduce aberrations. By requiring on the position of the dichroic mirror itself rather than an achromatic lens in order to reduce aberrations and achieve the desired distribution of light rays from each light source, the system remains relatively small and sufficiently lightweight for use in a freely moving rodent subject, such as a mouse or a rat.

In some embodiments, the system for in-vivo two color calcium imaging comprises a first light source and a second light source. In some embodiments, each light source is a single photon light source. In some embodiments, the system comprises a first light filter operably connected to the first light source to select for light of a first wavelength, and a second light filter operably connected to the second light source to select for light of a second wavelength. The first wavelength and the second wavelength may be any desired wavelength. The first wavelength and the second wavelength are different wavelengths, thus permitting detection of two different indicators present in the tissue of the subject.

The first wavelength and the second wavelength may depend on the calcium indicators present in the tissue of the subject. For example, in some embodiments the first wavelength is selected to excite a first calcium indicator and the second wavelength is selected to excite a second calcium indicator present in the tissue of the subject, thereby permitting simultaneous detection of two calcium indicators. In some embodiments, the first wavelength and the second wavelength are separated by at least 20 nm. In some embodiments, the first wavelength and the second wavelength are separated by at least 30 nm. In some embodiments, the first wavelength and the second wavelength are separated by at least 40 nm. In some embodiments, the first wavelength and the second wavelength are separated by at least 50 nm. For example, in some embodiments the first light filter selects for blue light having a wavelength of 450 nm-490 nm, and the second light filter selects for light having a wavelength separated from the first wavelength by at least at least 30 nm. Use of the wavelength 450 nm-490 nm would be useful for visualization of the calcium indicator GCaMP. In some embodiments, the first light filter selects for green light (e.g. lime) having a wavelength of 547 nm-572 nm, and the second light filter selects for light having a wavelength separated from the first wavelength by at least 30 nm. Use of the wavelength of 547 nm-572nmS would be useful for visualization of the calcium indicator jRCaMP. In some embodiments, the first light filter selects for blue light having a wavelength of 450 nm-490 nm, and the second light filter selects for light having a wavelength of 547 nm-572 nm. Such an embodiment would be useful for visualization of the calcium indicator GCaMP using the first wavelength, and the calcium indicator jRCaMP using the second wavelength. The above examples are in no way to be construed as limiting to the calcium indicators that may be used in conjunction with the system described herein or the filters to be used to visualize said indicators, and are only intended to exemplify how the first and second filters can be selected depending on the indicators present in the tissue.

In some embodiments, light energy transferred to the tissue from the first light source illuminates a first detectable signal from a first calcium indicator present in the tissue of the subject, and the light energy transferred to the tissue from the second light source illuminates a second detectable signal from a second calcium indicator present in the tissue of the subject. In some embodiments, the first detectable signal is captured by the first image sensor and the second detectable signal is captured by the second image sensor. In some embodiments, the portable system further comprises an achromatic lens and an emission dichroic mirror. In some embodiments, the first detectable signal and the second detectable signal each refract through the achromatic lens onto the emission dichroic mirror, and are subsequently split by the emission dichroic mirror, thereby reflecting the first detectable signal to the first image sensor and the second detectable signal to the second image sensor. The first image sensor and the second image sensor are housed in different locations within the system. In some embodiments, the first image sensor and the second image sensor are each complementary metal oxide semiconductor (CMOS) image sensors.

In some embodiments, the implanted lens is a gradient index (GRIN) lens. In some embodiments, the excitation lens is a plane-convex lens, a drum lens, a half ball lens, or a freeform lens.

In some embodiments, the system weighs about 5 g or less. In some embodiments, the system weighs about 4 g or less. In some embodiments, the system weighs about 3 g or less. In some embodiments, the system weighs about 5 g, about 4.9 g, about 4.8 g, about 4.7 g, about 4.6 g, about 4.5 g, about 4.4 g, about 4.3 g, about 4.2 g, about 4.1 g, about 4.0 g, about 3.9 g, about 3.8 g, about 3.7 g, about 3.6 g, about 3.5 g, about 3.4 g, about 3.3 g, about 3.2 g, about 3.1 g, about 3.0 g, about 2.9 g, about 2.8 g, about 2.7 g, about 2.6 g, about 2.5 g, about 2.4 g, about 2.3 g, about 2.2 g, about 2.1 g, or about 2.0 g. In some embodiments, the system weights about 2.5 g.

In some embodiments, the portable system is incorporated into a network. In some embodiments, the network comprises the portable system, along with one or more additional components that control one or more components of the portable system (e.g. control one or more lenses, control the single-photon light source, control the image sensor, etc.) and/or operate in conjunction with the portable system. For example, in some embodiments the network comprises the portable system, along with a computer. In some embodiments, the network comprises the system, along with a circuit board. In some embodiments, the network comprises the portable system, a computer, and a circuit board. In some embodiments, the circuit board loads data (e.g. calcium imaging data, behavioral recording data, data regarding the state of the portable system itself) to the computer. In some embodiments, the circuit board is operably connected to one or more components of the portable system (e.g. one or more mirrors, lenses, the light sources, etc.). In some embodiments, the circuit board is operably connected to the one or more components of the portable system in that it transmits signals to the one or more components of the portable system. For example, in some embodiments the circuit board transmits signals to the light source, thereby directing the operation thereof. In some embodiments, the circuit board transmits signals to one or more lenses, thereby directing the operation thereof. In some embodiments, the network comprises a behavioral data recording interface (BRDI). In some embodiments, the behavioral data recording interface comprises a camera, which records behaviors of the subject. In some embodiments, the BRDI comprises a camera, which records behavior of the subject while connected to the portable system (e.g. while performing calcium imaging on the subject). In some embodiments, the BRDI is operably connected to the circuit board, such that the circuit board can transmit signals to the BRDI to control the function thereof. In some embodiments, the circuit board additionally transmits data from the BRDI to the computer. In some embodiments, the computer executes a software program, such as a software program containing instructions to perform a given two-color calcium-imaging method.

In some embodiments, the system or network comprising the same is used in a method of in-vivo two-color calcium imaging in a freely-moving subject. In some embodiments, the subject is a rodent. In some embodiments, the system or network comprising the same is used in a method of in-vivo two-color calcium imaging in a freely moving mouse. In some embodiments, the method of in-vivo two-color imaging in a freely-moving subject comprises connecting the portable system to the subject, generating light rays from the light sources, and collecting data from the subject. In some embodiments, the method comprises connecting the portable system to the subject, generating light rays from the light sources (e.g. by executing a software program on the computer, which directs the circuit board to control the single-photon light source), thereby transferring light energy from first light source and the second light source to the illumination area of the subject. In some embodiments, the first light source and the second light source are integrated into a main excitation path by reflection from the excitation dichroic mirror onto the excitation lens, are subsequently refracted through the excitation lens onto the main dichroic mirror, and are then reflected by the main dichroic mirror onto the implanted lens, thereby transferring light energy from the first light source and the second light source to an illumination area on the tissue of the subject.

In some embodiments, the light energy transferred to the tissue from the first light source illuminates a first detectable signal from a first calcium indicator present in the tissue of the subject, and the light energy transferred to the tissue from the second light source illuminates a second detectable signal from a second calcium indicator present in the tissue of the subject. In some embodiments, the first detectable signal and the second detectable signal each refract through an achromatic lens onto the emission dichroic mirror, and are subsequently split by the emission dichroic mirror, thereby reflecting the first detectable signal to the first image sensor and the second detectable signal to the second image sensor. In some embodiments, the method further comprises capturing an image of the first detectable signal by the first image sensor, and capturing an image of the second detectable signal by the second image sensor. In some embodiments, the method further comprises converting the images to a readout of cellular activity. In some embodiments, the cells of the subject (e.g. cells within the illumination area) express or contain two different calcium indicators. In some embodiments, the cells express two different genetically encoded calcium indicators, such that the light rays generated from the first light source illuminates a detectable signal from a first indicator present in the tissue in the subject and the light rays generated from the second light source illuminate a detectable signal from a second indicator present in the tissue of the subject.

### 4. Portable Systems and Methods for Combined Modulation and Imaging of Cellular Activity In-Vivo

In some aspects, provided herein are portable systems and methods for combined modulation and imaging of cellular activity in-vivo. In some embodiments, provided herein is a portable system for combined modulation and imaging of cellular activity in-vivo. In some embodiments, the system comprises a spatial light modulator (SLM) which facilitates generation of a patterned excitation light path which is directed to the tissue of a subject, thereby inducing patterned modulation of cellular activity within a stimulation area on the tissue of the subject. In some embodiments, the system additionally comprises a single photon light source. In some embodiments, light energy from the single photon light source is transferred to an illumination area on the tissue of the subject. In some embodiments, the light energy transferred from the single photon light source to the tissue of the subject illuminates a detectable signal from a calcium indicator present in the tissue of the subject. In some embodiments, the illumination area and the stimulation area are substantially the same region within the tissue. Accordingly, the systems and methods for combined modulation and imaging of cellular activity in-vivo described herein permit patterned modulation of cellular activity and imaging of cellular activity within substantially the same region of tissue in a subject.

In some embodiments, the illumination area and the stimulation area are each are each substantially circular areas. In some embodiments, the illumination area and the stimulation area each have an average diameter of at least 150 microns. In some embodiments, the illumination area and the stimulation area each have an average diameter of at least 150 microns, at least 160 microns, at least 170 microns, at least 180 microns, at least 190 microns, at least 200 microns, at least 210 microns, at least 220 microns, at least 230 microns, at least 240 microns, or at least 250 microns. In some embodiments, the illumination area and the stimulation area each have an average diameter of about 250 microns. In some embodiments, the illumination area and the stimulation area are each substantially circular areas, wherein the overlap between the circular area of the illumination area and the circular area of the stimulation area is at least 90% (e.g. at least 90%, at least 91%, at last 92%, at least 93%, at least 94%, at least 95%, at least 96%, at least 97%, at least 98%, or at least 99% overlap). Accordingly, the systems described herein provide for a relatively large area (e.g. having a diameter of at least 150 microns) wherein both cellular illumination and stimulation occur. The systems described herein therefore generate a relatively large area wherein both precise patterned optogenetic modification of cellular activity and recording of cellular activity occur.

In some embodiments, the system comprises a laser, a collimation lens, and a spatial light modulator. In some embodiments, beams from the laser refract through the collimation lens to generated collimated laser beams, and the collimated laser beams are reflected by the spatial light modulator to generate a patterned excitation light path. In some embodiments, the spatial light modulator comprises a plurality of operably independent mirrors. In some embodiments, the collimated laser beams are reflected by the operably independent mirrors to generate a patterned excitation light path. In some embodiments, the patterned excitation light path is directed to the tissue of the subject, thereby inducing patterned modulation of cellular activity within the tissue.

In some embodiments, the system further comprises a main dichroic mirror and an implanted lens. Accordingly, in some embodiments the system comprises a laser, a collimation lens, a spatial light modulator, a main dichroic mirror, and an implanted lens. In some embodiments, the implanted lens is a GRIN lens. In some embodiments, beams from the laser refract through the collimation lens to generate collimated laser beams, and the collimated laser beams are reflected by spatial light modulator to generate a patterned excitation light path which is reflected by the main dichroic mirror onto the implanted lens, thereby transferring the patterned excitation light path to a stimulation area on the tissue of the subject and inducing patterned modulation of cellular activity within the stimulation area.

In some embodiments, patterned modulation of cellular activity comprises modulating the activity of a single target cell within the stimulation area. In some embodiments, patterned modulation of cellular activity comprises modulating activity of a plurality of target cells within the stimulation area. In some embodiments, patterned modulation of cellular activity comprises stimulating or enhancing the activity of a single target cell within the stimulation area. In some embodiments, patterned modulation of cellular activity comprises inhibiting the activity of a single target cell within the stimulation area. In some embodiments, patterned modulation of cellular activity comprises stimulating or enhancing the activity of a plurality of target cells within the stimulation area. In some embodiments, patterned modulation of cellular activity comprises inhibiting the activity of a plurality of target cells within the stimulation area. As described above, in some embodiments the illumination area is a substantially circular area having an average diameter of at least 150 microns (e.g. at least 150 microns, at least 160 microns, at least 170 microns, at least 180 microns, at least 190 microns, at least 200 microns, at least 210 microns, at least 220 microns, at least 230 microns, at least 240 microns, or at least 250 microns). Accordingly, the systems described herein achieve precise patterned modulation of cellular activity (e.g. patterned modulation of a plurality of cells, modulation of a single cell) for cell(s) within a relatively large window on the tissue.

In some embodiments, the system comprises a single photon light source, an excitation dichroic mirror, and an image sensor. In some embodiments, the system comprises a single photon light source, an excitation dichroic mirror, an excitation lens, a main dichroic mirror, and an implanted lens. In some embodiments, light rays from the single photon light source are reflected by the excitation dichroic mirror onto the excitation lens, refract through the excitation lens onto the main dichroic mirror, and are subsequently reflected by the main dichroic mirror onto the implanted lens, thereby transferring light energy from the single photon light source to an illumination area on the tissue of the subject. In some embodiments, the main dichroic mirror serves two purposes, namely a) reflection of the patterned excitation light path onto the implanted lens, and b) reflection of the light rays from the single photon light source onto the implanted lens. In some embodiments, the implanted lens is a GRIN lens. In some embodiments, the excitation lens is an achromatic lens.

In some embodiments, the light energy transferred from the single photon light source to the tissue of the subject illuminates a detectable signal from a calcium indicator present in the tissue of the subject. In some embodiments, an image of the detectable signal is generated by the image sensor. In some embodiments, the image sensor is a complementary metal oxide semiconductor (CMOS) image sensor.

In some embodiments, the system further comprises an excitation filter operably connected to the single photon light source to select for light of a first wavelength. For example, the excitation filter may filter out light of undesired wavelengths, thus selecting for light of the first wavelength (e.g. a desired wavelength). The first wavelength may be any desired wavelength and may depend on the indicator present within the tissue of the subject. For example, differing calcium indicators are illuminated in the presence of light of different wavelengths. Accordingly, the excitation filter may select for light of a first wavelength that illuminates a signal from a calcium indicator present in the tissue of the subject.

In some embodiments, the system further comprises an emission filter. In some embodiments, the emission filter constrains the emission light wavelength, thereby preventing disturbing light that may otherwise interfere with imaging. In some embodiments, the system further comprises an emission achromatic lens.

In some embodiments, the system comprises a laser, a collimation lens, a spatial light modulator (SLM), a single photon light source, an excitation dichroic mirror, an excitation lens, a main dichroic mirror, an implanted lens placed in a tissue of the subject, and an image sensor. In some embodiments: (A) beams from the laser refract through the collimation lens to generate collimated laser beams, wherein the collimated laser beams are reflected by the spatial light modulator to generate a patterned excitation light path which is reflected by the main dichroic mirror onto the implanted lens, thereby transferring the patterned excitation light path to a stimulation area on the tissue of the subject and inducing patterned modulation of cellular activity within the stimulation area; (B) light rays from the single photon light source are reflected by the excitation dichroic mirror onto the excitation lens, refract through the excitation lens onto the main dichroic mirror, and are subsequently reflected by the main dichroic mirror onto the implanted lens, thereby transferring light energy from the single photon light source to an illumination area on the tissue of the subject; and (C) the stimulation area and the illumination area are substantially the same region within the tissue of the subject.

In some embodiments, the system comprises a laser, a collimation lens, a spatial light modulator (SLM) comprising a plurality of operably independent mirrors, a single photon light source, an excitation dichroic mirror, an excitation lens, a main dichroic mirror, an implanted lens placed in a tissue of the subject, and an image sensor. In some embodiments: (A) beams from the laser refract through the collimation lens to generate collimated laser beams, wherein the collimated laser beams are reflected by the operably independent mirrors to generate a patterned excitation light path which is reflected by the main dichroic mirror onto the implanted lens, thereby transferring the patterned excitation light path to a stimulation area on the tissue of the subject and inducing patterned modulation of cellular activity within the stimulation area; (B) light rays from the single photon light source are reflected by the excitation dichroic mirror onto the excitation lens, refract through the excitation lens onto the main dichroic mirror, and are subsequently reflected by the main dichroic mirror onto the implanted lens, thereby transferring light energy from the single photon light source to an illumination area on the tissue of the subject; (C) the stimulation area and the illumination area are substantially the same region within the tissue of the subject; and (D) the stimulation area and the illumination area are each substantially circular areas having a diameter of about 250 microns.

In some embodiments, the system weighs 8 g or less. In some embodiments, the system weighs 7.5 g or less. In some embodiments, the system weighs 7 g or less. In some embodiments, the system weigh 6 g or less. In some embodiments, the system is used in a method of combined modulation and imaging of cellular activity in-vivo in a freely-moving subject. In some embodiments, the subject is a rodent. In some embodiments, the subject is a mouse. In some embodiments, the system is used in a method of in-vivo calcium imaging in a freely moving mouse.

In some embodiments, the portable system for combined modulation and imaging of cellular activity is incorporated into a network. In some embodiments, the network comprises the portable system, along with one or more additional components that control one or more components of the portable system (e.g. control one or more lenses, control the single-photon light source, control the image sensor, etc.) and/or operate in conjunction with the portable system. For example, in some embodiments the network comprises the portable system, along with a computer. In some embodiments, the network comprises the system, along with a circuit board. In some embodiments, the network comprises the portable system, a computer, and a circuit board. In some embodiments, the circuit board loads data (e.g. calcium imaging data, behavioral recording data, data regarding the state of the portable system itself) to the computer. In some embodiments, the circuit board is operably connected to one or more components of the portable system (e.g. one or more mirrors, lenses, the light source, etc.). In some embodiments, the circuit board is operably connected to the one or more components of the portable system in that it transmits signals to the one or more components of the portable system. For example, in some embodiments the circuit board transmits signals to the light source, thereby directing the operation thereof. In some embodiments, the circuit board transmits signals to one or more lenses, thereby directing the operation thereof. In some embodiments, the network comprises a behavioral data recording interface (BRDI). In some embodiments, the behavioral data recording interface comprises a camera, which records behaviors of the subject. In some embodiments, the BRDI comprises a camera, which records behavior of the subject while connected to the portable system (e.g. while performing calcium imaging on the subject). In some embodiments, the BRDI is operably connected to the circuit board, such that the circuit board can transmit signals to the BRDI to control the function thereof. In some embodiments, the circuit board additionally transmits data from the BRDI to the computer. In some embodiments, the computer executes a software program, such as a software program containing instructions to perform a given combined optogenetics and calcium-imaging method.

In some embodiments, provided herein are methods for combined manipulation and imaging of cellular activity in vivo. In some embodiments, provided herein is a method for combined modulation and imaging of cellular activity in vivo, comprising connecting a system described herein to a tissue of the subject. In some embodiments, the method comprises connecting the system to the tissue of the subject, generating beams from the laser, generating light rays from the one-photon light source, and obtaining an image of a detectable signal from the tissue of the subject.

In some embodiments, the method comprises: (A) generating beams from the laser, thereby transferring a patterned excitation light path to a stimulation area on the tissue and inducing a patterned manipulation of cellular activity for one or more cells within the stimulation area, and (B) generating light rays from the one-photon light source, thereby illuminating a detectable signal from an indicator present in the tissue of the subject. In some embodiments, the method comprises obtaining an image of the detectable signal. In some embodiments, the method further comprises converting the image of the detectable signal to a readout of a cellular activity pattern of one or more cells in the subject.

In some embodiments, the cells of the subject (e.g. cells within the illumination area) express or contain a calcium indicator. In some embodiments, the cells express a genetically encoded calcium indicator, such that the light rays generated from the light source illuminate a detectable signal from the indicator present in the tissue in the subject. In some embodiments, the subject expresses one or more light-sensitized proteins, such that optogenetic modulation of cellular activity is possible. For example, the cells of the subject may express one or more light-activated proteins (e.g. opsins). Any suitable light-activated protein may be used, including light-activated channels (e.g. light-activated potassium channels, light-activated ion pumps, etc. In some embodiments, the light-activated protein is a halorhodopsin, channelrhodopsin, enhanced halorhodopsin, archaerodopsin, fungal opsin, or enhanced bacteriorhodopsin. In some embodiments, the light-activated protein is a channel rhodopsin (e.g. ChR2, ChR1, VChR1, SFO, etc.).

In some aspects, provided herein are methods of mimicking cellular activity. In some embodiments, provided herein is a method of mimicking cellular activity in a subject, comprising manipulating and recording cellular activity in-vivo as described herein, and selectively targeting one or more cells with a patterned excitation light path to reproduce the activity of the cells previously recorded. In some embodiments, mimicking the cellular activity pattern of the one or more cells comprises selectively targeting the one or more cells with the patterned excitation light path, such that a patterned modulation of activity for the one or more cells is induced.

### EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

One-photon calcium imaging with endoscope is a technique used for calcium imaging of large populations neurons in awake animals. However, in current systems for one-photon calcium imaging, including the Miniscope, a half ball lens is used to collect LED emitting energy. This half ball lens has no light energy distribution functions, so when the LED turns on, there is an unequal energy distribution with peak illumination in the center of the illumination area and substantially reduced illumination towards the edge of the illumination area. This unequal energy distribution causes substantial problems. Specifically, (1) the calcium fluorescence brightness in the center is so strong that it will cover the weaker fluorescence of surrounding neurons. (2) because of the non-uniform energy distribution, the field of view will be smaller than expected, and (3) the neurons in the center are susceptible to photo bleaching than surrounding neurons.

This example provides an exemplary portable imaging system that addresses the above issues. The system described herein is a lightweight and portable imaging system that can be used in freely moving animals. The system achieves a substantially even distribution of light rays relative to a center point within the illumination area, and is thus advantageous over other systems for one-photon imaging.

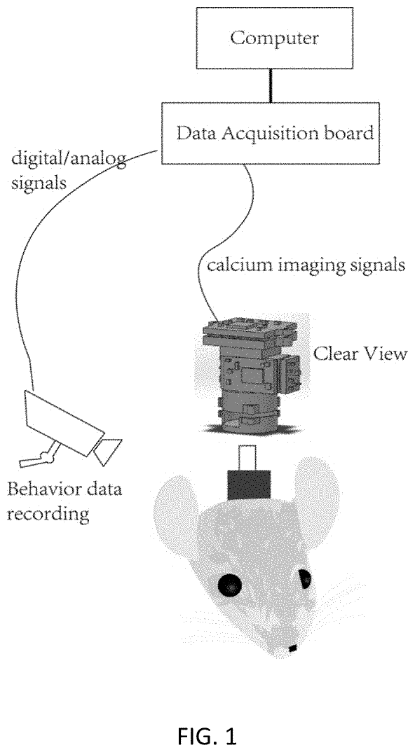

The exemplary portable system described in this example is incorporated into a network, referred to as “Clear View”. An exemplary network comprising the portable system described herein is shown in FIG. 1. As illustrated in FIG. 1, the network comprises a portable system for in-vivo imaging as described herein, referred to as the “Clear View Body” or “CVB”. The network further comprises a circuit board, referred to herein as the “Data Acquisition Board” or “DAB”, a behavior data recording interface (BDRI), and Data Recording Software (DRS). The network further comprises a computer.

The DAB is an electronic PCB board to upstream load all the data flow (calcium imaging data flow, behavior recording data, state of the clear view body) to the computer, and transmit control signals (LED control, starting signal control, electrically tunable lens (ETL), etc.) to the CVB and BDRI. The CVB is the main part of the network, it contains the portable system described herein in a small and light weight body. In this example, the CVB contains the CMOS (the image sensor), an LED control subsystem, an ETL control subsystem, and information exchange subsystem.

The BDRI is an extended connector, for customized recording of behavior data. It supports 16 digital I/O ports and 1 behavior camera port. The data from the BRDI upload to the DAB, and the DAB uploads to the computer. The DRS display all the data flow and signals to a user, and the user can control all the parameters in the DRS.

There is a synchronous acquisition clock in DAB. This clock is used to synchronize the data flow and behavior signals in the same timeline.

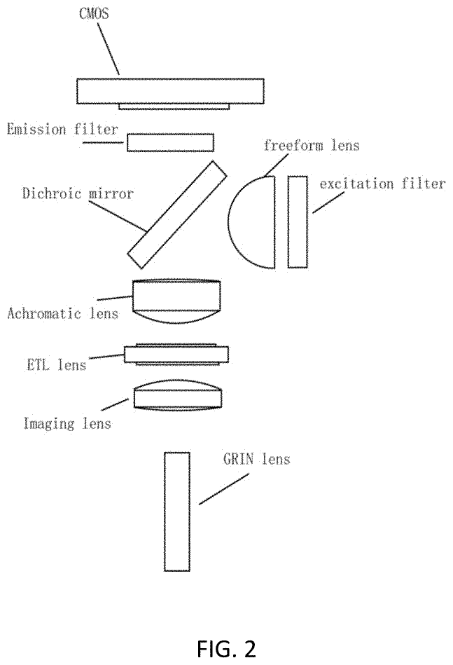

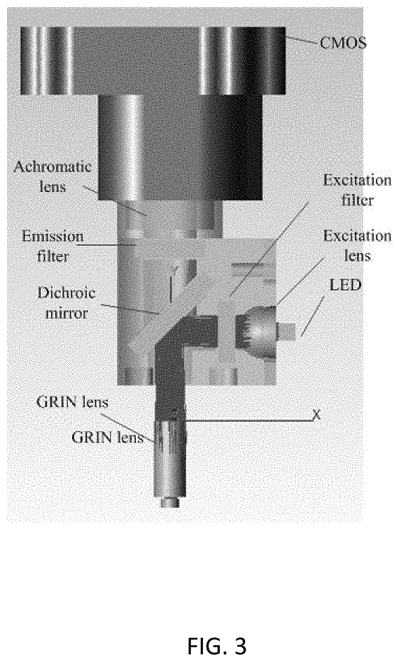

The CVB comprises an excitation filter, emission filter, dichroic mirror, freeform lens, ETL, imaging lens and GRIN lens, along with an image sensor. A diagram of components of an exemplary CVB is shown in FIG. 2. A diagram of the components of the exemplary CVB when placed together is shown in FIG. 3. The excitation filter is for constraining excitation light wavelength. For example, if a GCaMP6s virus was injected in the mice brain, then the excitation filter should be blue light (450-490 nm) to excite virus. If jRCaMP1b virus was injected in the mice brain, then the excitation filter should be orange light (550-590). The filters can be replaced depending on the viruses. The wavelength of excitation filter, emission filter, dichroic mirror should be separated at least 10 nm with each other, to avoid crosstalk. The emission filter is for constraining the emission light wavelength, so that there is no other disturbance light in the calcium imaging. The achromatic lens, ETL lens and imaging lens are chosen for generating clear image on the CMOS. The focal plane can be changed when adjusting ETL lens. The GRIN lens is a cylinder lens which can be implanted in the brain. Different diameters and lengths for the GRIN lens can be chosen depending on the intended tissue to be imaged. For example, different diameters and lengths for the GRIN lens may be chosen for different brain areas.

### Freeform Lens Design

Considering the tiny size of Clear View body, the required energy distribution on the dichroic mirror was analyzed, and the freeform lens was designed based on the point light source (PLS). For this example, the point light source was assumed to be an LED light source. Then, a freeform lens surface was designed, and the energy feedback freeform lens (EFFL) method was used to optimize the energy distribution to decrease the error based on the PLS assumption. Compared to the half ball lens traditionally used in existing calcium imaging systems, the freeform lens collects twice more energy and the energy distribution in the center is significantly more equal. Moreover, because of the equal energy distribution achieved using the freeform lens, center photo bleaching can be reduced, allowing for more sustained experiments to be performed.

The maximum diameter of the excitation lens is 3 mm. An LED having a size of 1*1 mm was regarded as an Extended light source (ELS). Here, the freeform lens was designed assuming that the point light source (PLS) is an LED. The basis for PLS illumination designs is determined entirely by the edge rays, independent of the curvature of the lens.

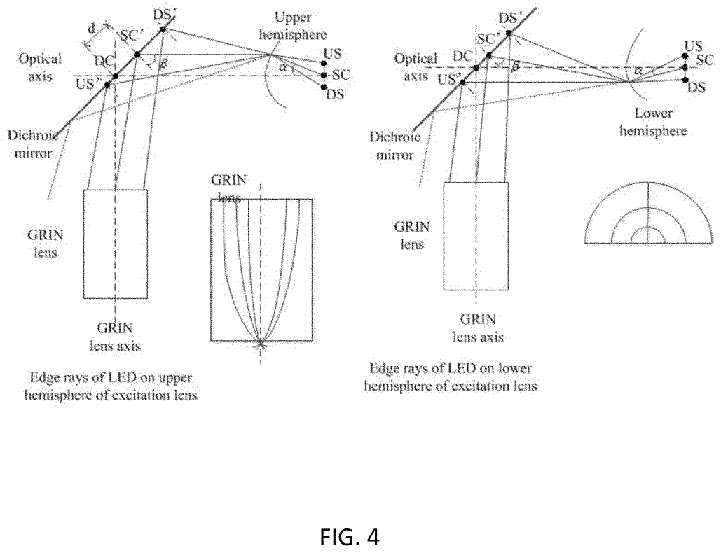

For the upper hemisphere of the excitation lens, as shown in FIG. 4, the edge rays of LED marked US and DS refracting through the excitation lens radiate on the dichroic mirror at US' and DS′, and reflected rays of US' and DS' all radiate on the surface of GRIN lens, which means most of the LED energy will be transferred into brain. In contrast, most of energy will be transferred out of GRIN lens surface if the center of LED rays (SC) radiate on the half bottom of the dichroic mirror (below DC). As the magnitude of 9 (the angle between SC ray and the optical axis) increases, the SC′ increases to the top half of dichroic mirror, which causes edge rays US' and DS' to reflect in the GRIN lens range. For the lower hemisphere of the excitation lens, as shown in FIG. 4, as the magnitude of θ increases, the SC′ increases to the top half of dichroic mirror as well. Note that the distance between SC′ and DC is smaller than the upper hemisphere because β (the angle of incidence on dichroic mirror) is smaller.

From the tailored edge ray analysis, the probable prescribed illumination pattern on the dichroic mirror can be described with θ and d. And because of axial symmetry of GRIN lens, the illumination pattern on dichroic mirror can be also treated as an axial symmetry half circle, which can simplify the energy mapping setting and freeform surface calculation to equation 1:

d=κθ (1)

Where κ is a coordinate factor.

Then, 10 points were set on the generatrix, and every point was determined by equation 1. The incident light from the PLS refracts on the plane surface and then towards the dichroic mirror. The edge light reflected by the dichroic mirror radiates on the edge of the GRIN lens, and then the point normal vector and space location can be calculated by Snell's law, equation 2:

[1+n²−2n(\vec{O}ᵢ·\vec{I}′ᵢ)]^{1/2}\vec{N}ᵢ=\vec{O}ᵢ−n\vec{I}′ᵢ (2)

Once all the points on the generatrix were calculated, a B-spline curve was used to fit all the points to generate a smooth generatrix. A rotating surface was generated by rotating with the axis, and a lens was generated by combining with the first plane surface.

Once the lens was generated, a simulation was performed based on Monte Carlo ray-tracing using Tracepro. Given that the GRIN lens concentrates light on a point if given parallel light, the illumination distribution is equal before going into the GRIN lens. Since the energy beneath the GRIN lens appears to be focused at the center of the light spot, if the energy mapping is based on a bright center and dark edges, the illumination distribution because of the GRIN lens deviation should be corrected to equal. Therefore, to solve the error from the PLS assumption, E=f(y) was selected as the deviation correction function of the required energy distribution, and was added into the edge light calculation, which is shown in equation (7).

$$\frac{{\int }_{{y}_{1}}^{{y}_{2}} 2 π {\sum }_{i = 1}^{n} {k}_{i} {f}_{i} ( y ) ydy}{{\int }_{0}^{R} 2 π {\sum }_{i = 1}^{n} {k}_{i} {f}_{i} ( y ) ydy} = \frac{{\int }_{{θ}_{1}}^{{θ}_{2}} 2 π I ( θ ) sin θ d θ}{{\int }_{0}^{{θ}_{MAX}} 2 π I ( θ ) sin θ d θ} \qquad ( 7 )$$

Where kᵢ is the weighting coefficients for every time the function added.

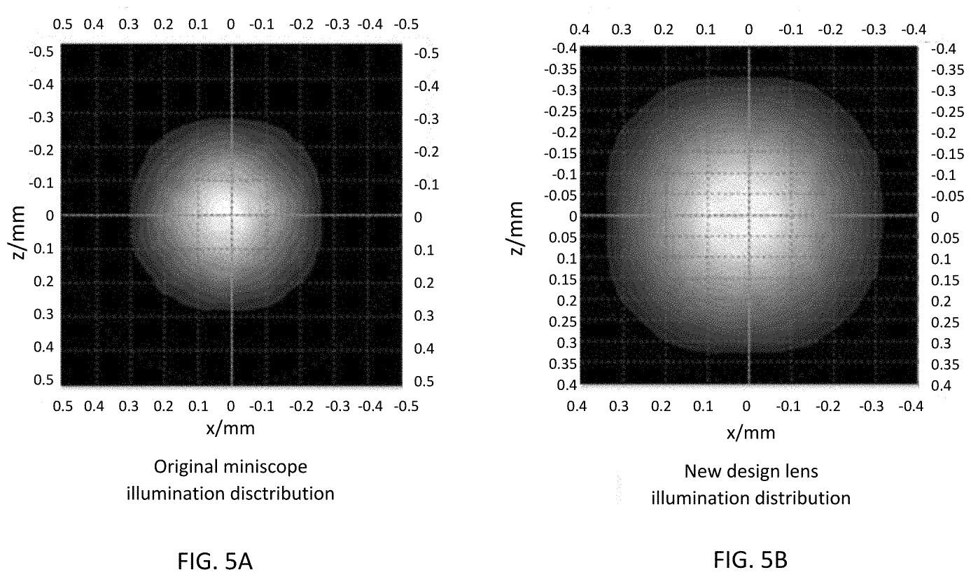

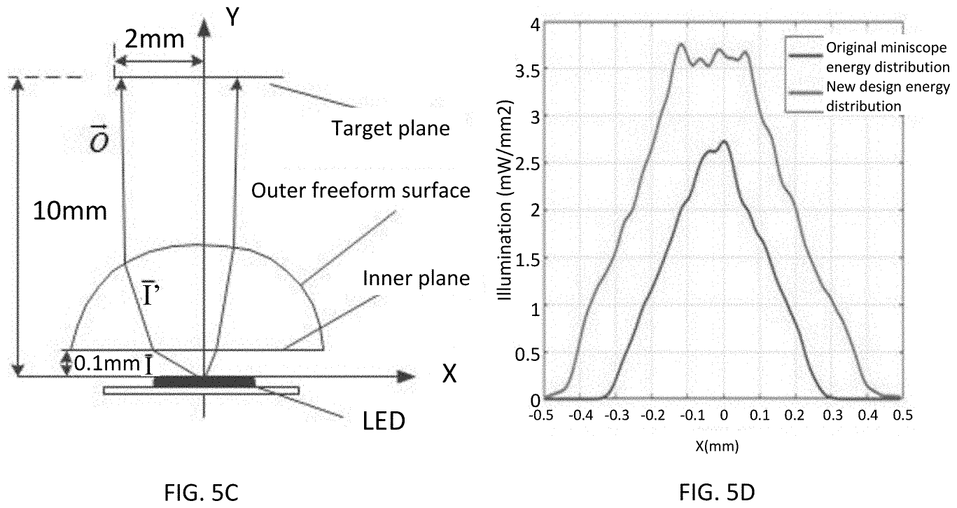

The freeform lens is far superior to the half ball lens commonly used in calcium imaging systems. The freeform lens can collect twice as much energy as a half ball lens, with more equal energy distribution. Consequently, the fluorescence field is bigger than standard designs, with no more center brightness overlap. This is shown in FIG. 5.

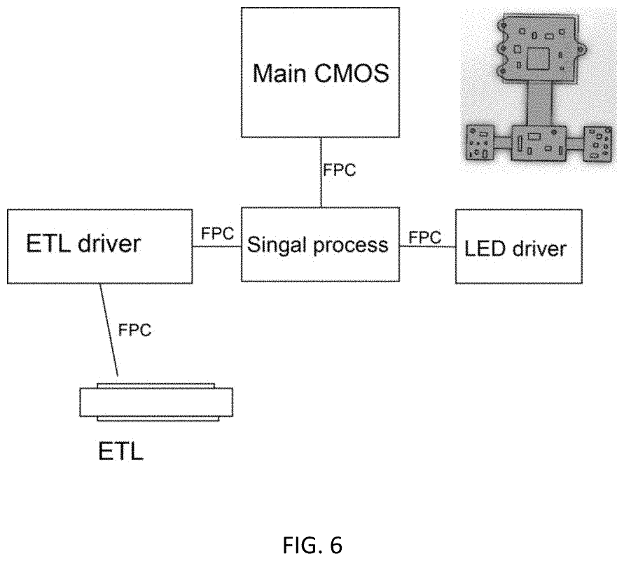

In this example, the image sensor is a CMOS. The CMOS (FIG. 6) comprises four sub-boards: a main CMOS board, a signal process board, an LED driver board, and an ETL driver board. The main CMOS board collects fluorescence images from the bottom side and the image data is processed in the driver IC and coaxial cable IC on the top side. This board transmits video data flow to the data acquisition board (DAB) and simultaneously receives control data from the DAB.

The signal process board connects with the main CMOS board by an FPC connector. The main function of this board is separating high-frequency signals and direct current and supporting power to ICs, LED and ETL. There are three kinds of power in CMOS, 1.8V, 3.3V, and 5V. LED driver board supplies power for LED, and the power can be also changed by the control signal. The ETL locates on the other side of the signal process board. It supplies the power and driver for the ETL, so the diopter can be adjusted by the control signal.

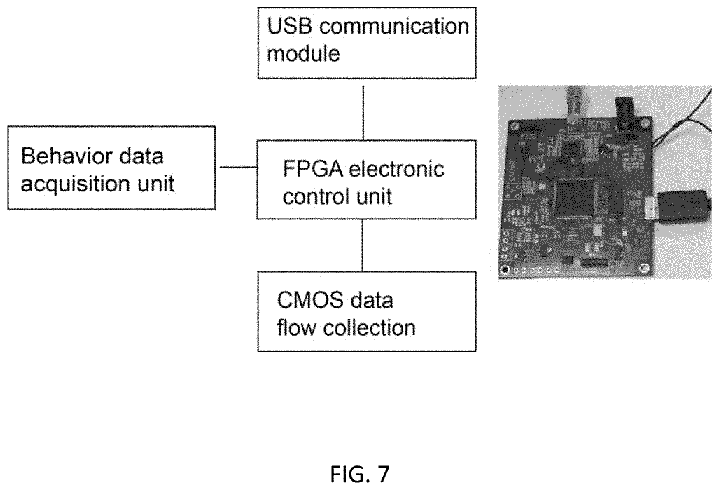

In this example, there is only one board for the DAB (FIG. 7), but it contains multiple functional areas including CMOS data flow collection, USB communication module, FPGA electronic control unit and behavior data acquisition unit. The FPGA electronic control unit processes multiple signals. It transmits the CMOS data flow to USB communication module immediately when it receives them from CMOS; and all the control signals from computer are decoded in this unit and sent to different ports. There is also a timer in the FPGA to synchronize the frame signals and the behavior data.

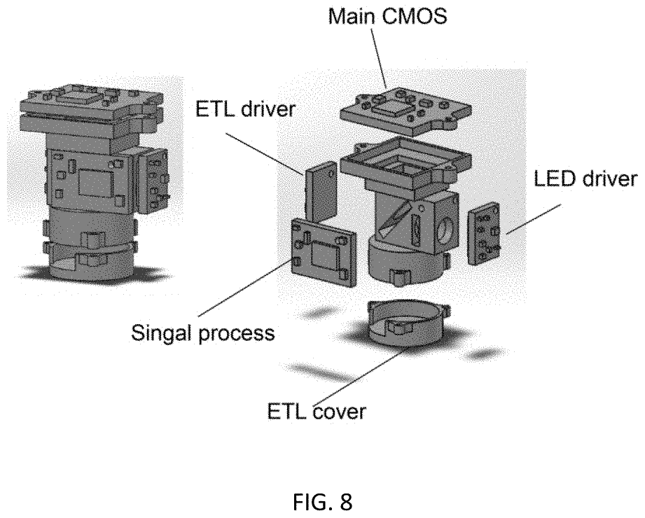

All of the components of the clear view body are assembled in a tiny body (FIG. 8). In this example, the main CMOS board is fixed on the top of the body, and the signal process board is fixed on the side of the body. The signal process board covered with black epoxy resin. When it fixed on the side, it covers the excitation filter and dichroic mirror. The LED driver board is fixed on the narrow side of the body, and when it fixed, the freeform lens can be also fixed in front of it. The ETL is fixed at the bottom of the main body, and is connected with the ETL FPC. The entire Clear View Body weighs about 2.5 g. This tiny size and weight permits relatively small animals like mice to carry it during behavioral experiments without notable hinderance to their movement or natural behavior.

### Example 2

This example provides a portable system for two-color imaging. The two-color imaging system is a portable, lightweight system that allows for simultaneous recording from two different populations of cells in freely moving subjects, including relatively lightweight subjects such as rodent (e.g. mice).

Currently, the only method for recording two color calcium imaging (Inscopix nVue) uses a single CMOS with alternative excitation of two indicators and recording from two channels. However, with only one emission filter and one CMOS, the crosstalk between the two indicators cannot be sufficiently distinguished. Attempts to distinguish between the two indicators rely on after imaging processing. After imaging processing can cause errors, for example due to slow decay of fluorescence from one indicator which can interfere with detection of the other indicator in the next frame recording.

Described herein is a novel system for two color calcium imaging that resolves these issues at least in part by using two separate CMOS sensors, while still providing for a system that is sufficiently lightweight to enable use in a freely moving mouse. In this novel design, the addition of a dichroic mirror in the emission path splits two fluorescence paths onto two independent image sensors. Two different wavelength LEDs are used to excite two indicators, and the two CMOS can record two channels at the same time.

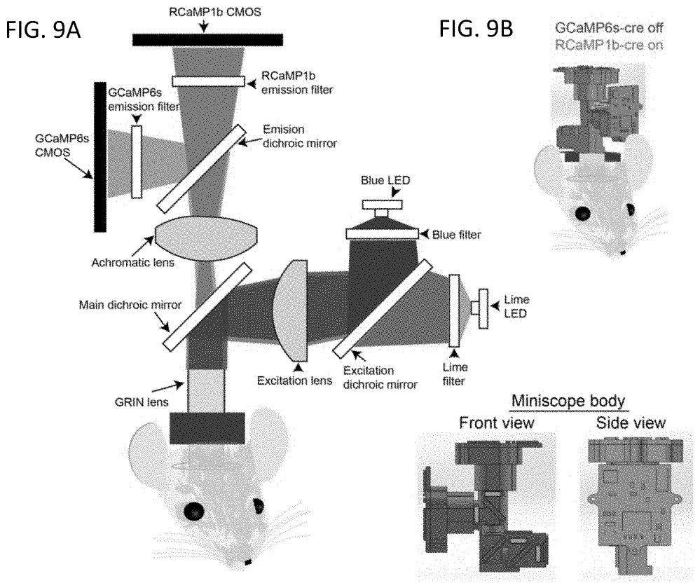

An exemplary system for two-color calcium imaging is shown in FIG. 9. As shown in FIG. 9, the system (also referred to as an endoscope) comprises an excitation path and emission path. For the excitation path, two different wavelength LEDs are fixed on different location on the endoscope body. In this example, one is blue LED (450 nm˜490 nm filter) for GCaMP calcium indicator excitation, and the other is lime LED (547 nm˜572 nm filter) for jRCaMP excitation. However, it is understood that other wavelengths of light may be used, and that the wavelength can be selected to function with any desired calcium indicator, including GECIs and chemical indicators.

In this example, the light sources (e.g. the LEDs) are controlled by a circuit board, referred to herein as the data acquisition board (DAB). The blue LED is controlled by a constant source capable of producing 30 mA with step of 1 mA, while lime LED is controlled by a designed constant source capable of producing 200 mA with step of 4 mA. The power sources can be controlled by the circuit board (e.g. the DAB). Both power sources are with up to 2 mW/mm2 measured beneath gradient-index (GRIN) lens. Two wavelengths of light are integrated in the main excitation path by reflection from the excitation dichroic mirror (T510lpxr). For the emission path, the fluorescent signals from the two indicators are split by an emission dichroic mirror and radiate into different CMOS. An achromatic lens is used in the main emission path to make sure the imaging is clearly reached on the CMOS.

The fluorescence field of the indicator is relevant to the illumination area under GRIN lens. Additionally, the illumination area for each color indicator should be the same to ensure that the same area of the calcium fields is being measured. The optical axis of blue and lime LED should be the same. To concentrate enough energy to excite the indicators, a plane-convex lens is used in the excitation path. But the two color illumination area, because of the chromatic and spherical aberration, will not overlap very well without intervention. To solve this problem, the excitation dichroic mirror was moved up 0.25 mm to reduce aberration. An achromatic lens was not used to eliminate aberrations, as it would increase the weight and volume of the system and render it unusable for small subjects such as mice. To test this design, a simulation was performed in Tracepro software. The simulation shows that the illumination areas beneath the GRIN lens were perfect overlapped. A test of the two-color system under an optical resolution chart further demonstrated that the two fluorescent fields perfectly overlapped with one another.

### Example 3

Optogenetics and calcium imaging can be combined to simultaneously stimulate and record neural activity in vivo. However, this usually requires two-photon microscopes, which are not portable or affordable. This example describes the design and implementation of a miniaturized one-photon endoscope for simultaneous optogenetic stimulation and calcium imaging in freely moving mice. The system is also referred to in this example as “Miniscope with All-optical Patterned Stimulation and Imaging” or “MAPSI”. By integrating a spatial light modulator into the portable system, it is possible to activate any cell of choice within the field of view or to apply arbitrary spatiotemporal patterns of photo-stimulation, while imaging calcium activity at the same time. In the striatum, this system was used to select specific direct or indirect pathway neurons on the basis of their relationship with a specific behaviour, and to recreate the behaviour by mimicking the natural neural activity with photostimulation. Accordingly, the portable system for combined modulation and imaging of cellular activity (e.g. the miniaturized endoscope for all-optical patterned optogenetic stimulation and calcium imaging) provides a powerful tool for in vivo interrogation of neural circuits. Moreover, MAPSI integrates pattern stimulation and calcium recording in a single system with a small body (25 mm×15 mm×15 mm), thus enabling use in freely moving relatively lightweight subjects, including freely moving mice. MAPSI makes it possible to mimic natural physiological patterns just recorded, instead of delivering artificial stimulation patterns to all neurons expressing opsins indiscriminately. Neural activity can be recorded and analysed in real-time while selecting and stimulating neurons that are behaviourally relevant. MAPSI thus makes it possible to perform closed loop experiments in which neural activity is maintained or shaped online.

### Results

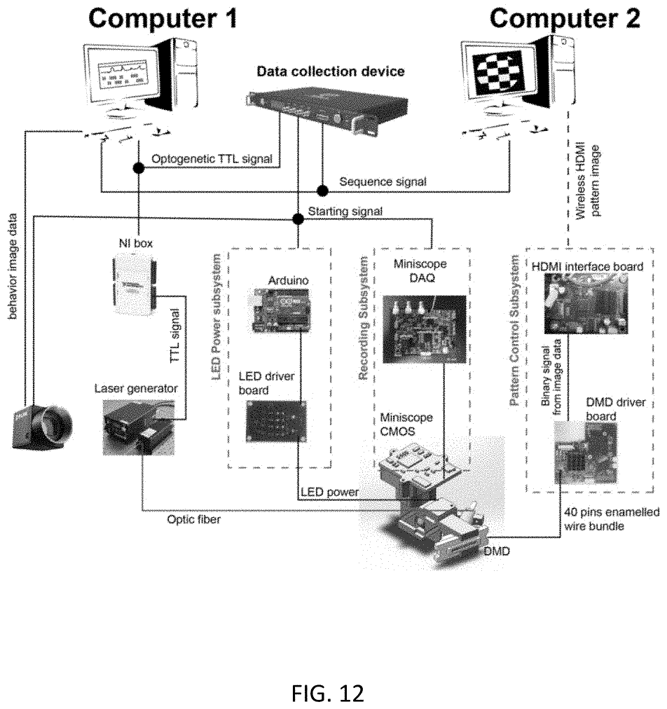

Design of MAPSI. Spatial light modulators, such as digital micromirror devices (DMDs), can create arbitrary light patterns with extremely high spatial and temporal resolution. To record and stimulate simultaneously, the MAPSI system uses two light sources, the first light source for excitation of calcium indicators and the second light source is a laser for optogenetic excitation. In this particular example, the first light source is a lime LED (540 nm-580 nm filter) for excitation of calcium indicators. This LED is controlled by a constant current source capable of producing up to 200 mA with a step size of 4 mA, with up to 12 mW/mm² measured beneath the gradient-index (GRIN) lens (FIG. 12). The second light source is an external laser (Opto Engine LLC, PSU-H-LED) that generates blue light for optogenetic excitation (473 nm). By passing the excitation light through the DMD, a patterned light beam is generated, which is merged with the excitation light for calcium imaging in the main excitation path (FIG. 14A-B). The DMD is controlled by a display controller (DLP3430) that can connect to any computer through an HDMI interface board (FIG. 12). The orientation of the micromirrors can be precisely controlled by a computer to create different stimulation patterns and sequences. It can be programmed to split the collimated light from the laser into individual beams, allowing the experimenter to select and stimulate extremely small areas (FIG. 13A-B & FIG. 10). In the DMD off-state, an absorber is used to avoid light dispersion into the excitation light path.

To obtain high resolution, a challenge is to produce almost perfectly collimated light (<1% deviation). The design was first simulated in optic tracing software (FIG. 11). In the main excitation light path, the focal planes of the achromatic lens and the GRIN lens are matched so that the light will precisely target specific regions beneath the GRIN lens. With this collimated beam, photo-stimulation can maintain its precise beamlets with high resolution even after traversing long distances (20 mm) through the excitation path and the GRIN lens.

Because MAPSI weights 7.8 g, to help mice to carry it a commutator with a pulley was developed to reduce the weight carried by 4 g, thus allowing free movement for long periods.

Functional capabilities of MAPSI. To test the ability of MAPSI to simultaneously stimulate and record neurons in freely moving animals, viral vectors containing Cre-dependent jRCaMP1b and ChR2-eYFP were injected in the dorsolateral striatum (DLS). jRCaMP1b is excited by a lime LED with a 540-580 nm excitation filter, and ChR2 is activated using an excitation wavelength of 473 nm with a 450-490 nm emission filter from a separate laser generator. Using Cre-dependent viral vectors (AAV1-CAG-Flex-jRCaMP1b and AAV5-EF1aa-DIO-hChR2-eYFP) in D1-Cre and A2A-Cre mice, jRCaMP1b and ChR2-eYFP were expressed in direct (D1+) and indirect (A2A+) pathway neurons.

MAPSI was first tested in anesthetized mice to validate that the stimulation area was similar to the calcium imaging region (FIG. 13A). A baseplate was used to fix MAPSI on the mouse head. More neurons can be recorded and stimulated by re-baseplating to cover another region beneath the GRIN lens. Using a 1.8 mm GRIN lens, the recording FOV is circular, with a diameter of ˜250 μm. The power density is 2 mW/mm² for imaging excitation, and 60-80 mW/mm² for optogenetic stimulation. In MAPSI, with a spot 10 μm in diameter for single neuron stimulation, the power is ˜60-80 mW/mm², which is unlikely to create considerable tissue heating.

Axial and lateral resolution. To determine the axial (z-axis) resolution of MAPSI, fluorescence signals were first recorded from a 200 μm brain slice from a D1-Cre mouse infected with ChR2 and RCaMP1jb using a confocal microscope, while simultaneously stimulating neurons using MAPSI. The axial resolution and depth of penetration are shown in FIG. 13B. The full-width half-maximum (FWHM) of the stimulation beamlets is approximately 30 μm. The same experiment was also performed in a brain slice from an A2A-Cre mouse, with similar results.

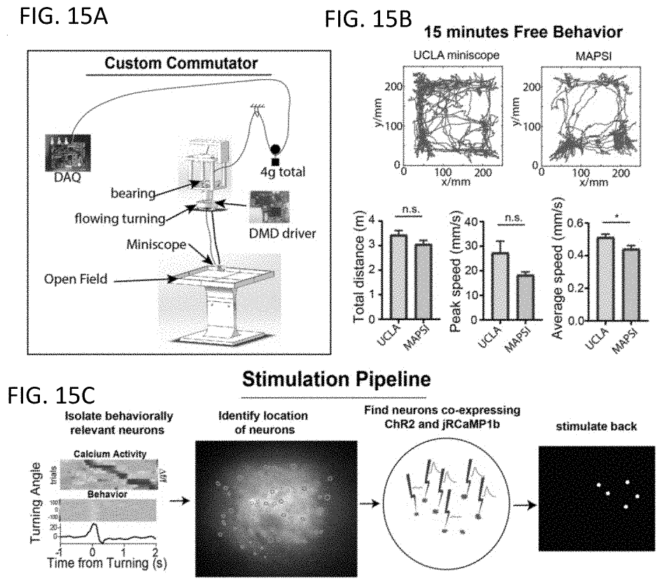

When the laser spot is 10 μm in diameter, the fluorescence detected was almost circular with 10.5 μm FWHM on the x-axis and 9.8 μm FWHM on the y-axis (FIG. 13C). The fluorescence linearly increased as the diameter of the illuminated area increased, and the intensity decreased as the beamlets penetrated more deeply into the tissue (FIG. 13D). To identify which neurons co-expressed both ChR2 and jRCaMP1b, the jRCaMP1b was continuously excited at low power (˜1 mW/mm²) to achieve a stable baseline, and ChR2 was excited at 20 Hz (20 ms pulse duration, 20 pulses, power density ˜50 mW/mm²). During stimulation, a significant increase in jRCaMP1b fluorescence signal was measured by targeting the neuron using a 10 μm diameter spot (FIG. 13E, 31 neurons in 6 mice. All traces are shown in FIG. 15. In the absence of ChR2 expression, no fluorescence change was detected (FIG. 13E; 21 cells from 4 control mice with only jRCaMP1b expression).

Testing in freely moving mice. MAPSI makes it possible to select individual neurons that are active during behaviors of experimental interest and play back their activity. Cre-dependent viral vectors and Cre driver lines were used to target either the direct pathway (striatonigral, D1-cre) or the indirect pathway (striatopallidal, A2A-cre). These two pathways have opposite effects on basal ganglia output and behaviour. In particular, stimulation of direct pathway spiny projection neurons (dSPNs) can produce contraversive turning behaviour (away from the side of stimulation, toward the contralateral side), and stimulation of indirect pathway spiny projection neurons (iSPNs) can produce ipsiversive turning behaviour (toward the side of stimulation). Thus, optogenetic manipulation of these pathways provide convenient behavioural readouts to test the efficacy of our all-optical stimulation/imaging system.

In order to mimic naturally occurring neural activity patterns, neurons that were active during a behaviour of interest were identified. If they co-expressed both ChR2 and jRCaMP1, optogenetic stimulation of these neurons was performed while recording their activity (FIG. 12).

MAPSI was tested in D1-Cre mice or A2A-Cre mice, with a chronically implanted GRIN lens (1.8 mm diameter, 4.3 mm length) during freely-moving behaviour. Multiple 10 μm beamlets (5 beamlets at 80 mW/mm² for dSPNs, 4 beamlets at 60 mW/mm² for iSPNs) were generated for stimulation while recording calcium activity from all neurons in the FOV. Individual dSPNs or iSPNs could be robustly and selectively activated without activating neighboring neurons (FIG. 16). Occasionally a non-stimulated neuron (e.g. neuron 6 in FIG. 16C) located close to a stimulated neuron (neuron 1 in FIG. 16C) responded almost systematically but with a longer latency (FIG. 16D). This suggests that some neurons can be activated indirectly by the stimulation via circuit connections.

Isolating Behaviorally Active Neurons and Manipulating their Activity

Isolating behaviorally active neurons and manipulating their activity. To examine the effects of direct pathway activation, Cre-dependent jRCaMP1b and ChR2-eYFP were injected into D1-Cre mice (N=3 mice) and only jRCaMP1b in control mice (N=2 mice) while simultaneously recording their behaviour in an open field arena (FIG. 17A). D1-neurons increase their activity during contraversive movements, as such neurons that increased firing within 500 ms from the start of contraversive turning movement were classified as turning-related neurons (28 out of 86 recorded neurons from 3 mice, 6-22 trials per mouse). The turning angle was computed after labeling each frame in DeepLabCut⁴⁰ (FIG. 17B-C). First, DeepLabCut was used to mark two points, one on the head and one on the back of the mouse, these points were used to generate a ‘head-body’ vector. Two successive frames (50 FPS) were compared to calculate the change in vector angle, which is used as a measure of turning. The derivative of the headbody vector was used as a measure of the angular deviation of body posture, regardless of whether the mouse was walking or not. 15 of the turning-related neurons (5 neurons in each mouse) were selected for stimulation (representative mouse shown in FIG. 17D). Selective stimulation of these neurons elicited turning that was comparable to the mice's natural turning (FIGS. 17G & H, compare to 17B & E). Surprisingly, stimulating 5 neurons was sufficient to produce contraversive turning. Different parameters of stimulation can produce contraversive turning. (FIG. 13B). The magnitude of the behavioural effect depended on the number of neurons stimulated (FIG. 13). In contrast, stimulating 5 neighboring neurons that are not related to turning did not produce significant turning (FIG. 17G-H, D1-cre mice, 25 trials for stimulated neurons, 20 trials for neighboring neurons; 16 trials in 2 control mice).

The same experiment was performed with A2A-Cre mice (N=3 jRCaMP1b and ChR2 mice, N=2 controls with only jRCaMP1b) (FIG. 18A). Activation of the indirect pathway neurons is known to produce ipsiversive turning³⁵. Out of 354 recorded iSPNs, 36 were related to ipsiversive turning. 12 neurons (4 neurons in the FOV in each mouse) that showed robust excitation during ipsiversive turning were selected (N=3 mice; FIG. 18B-C). These turning-related neurons (n=12 neurons from 3 mice) were then excited, which produced ipsiversive turning (FIG. 18E-G). The magnitude of the turning effect depended on the number of neurons stimulated (FIG. 16). Different parameters of stimulation can also produce ipsiversive turning (FIG. 17). Neurons that were not active during turning were also stimulated, which did not produce significant ipsiversive turning.

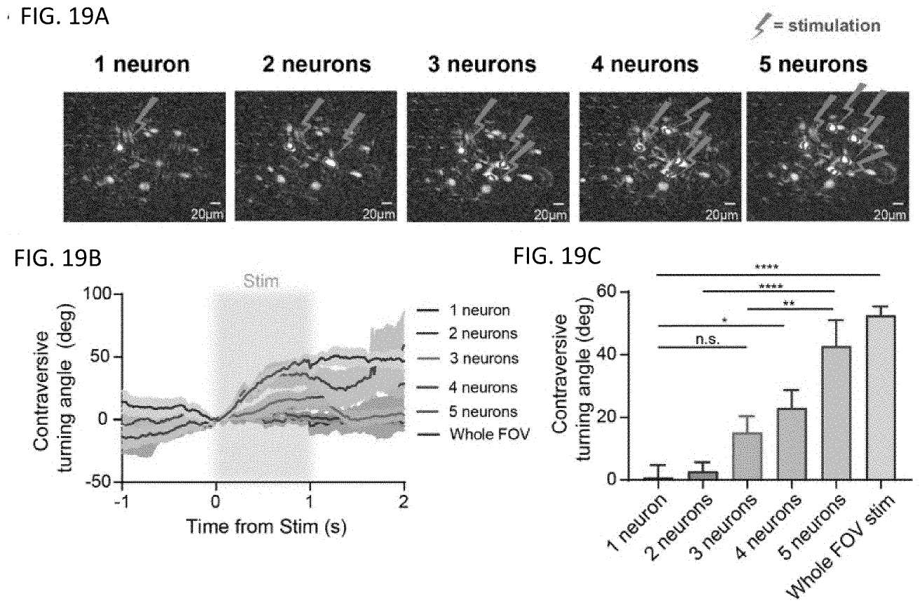

Moreover, the effect of selective stimulation was also stable across time. A specific ensemble of neurons was selectively stimulated on one day, the behavioural effect was replicated by stimulating the same neurons 40 days later (FIG. 19, 2 A2A-cre mice). There was no significant difference in the ipsiversive turning that was elicited on day 1 compared to day 40.

Synthesizing sequences and sweeping patterns of stimulation. To test the effect of arbitrary stimulation patterns, two sequential patterns were used to activate turning-related neurons: either lateral to medial (LM, starting with the most lateral neuron) or medial to lateral (ML, starting with the most medial neuron) (FIG. 20B-C). Both sequences produced contraversive turning (FIG. 20D).

MAPSI also makes it possible to produce arbitrary spatiotemporal patterns of light to sculpt neural activity. The DMD was programmed to produce rectangular sweeping patterns that covered ˜20% of the FOV. Two different directions (ML or LM) across the FOV were used in both hemispheres (FIG. 20F). In D1-Cre mice (N=3) either pattern produced contraversive turning, but there was no significant difference between different sweep directions (ML or LM) regardless of hemisphere stimulated (FIG. 20G, H).

The same experiments were performed using A2A-Cre mice (FIG. 21A). Neurons that were active during ipsiversive turning were identified, the same sequential patterns as used in FIG. 6 (FIG. 21B) were used. The calcium activity was also verified during the sequential pattern stimulation (FIG. 21C). Both sequences produced ipsiversive turning with the LM sequence (#1) producing greater ipsiversive turning (FIG. 21D). The sweeping patterns used in FIG. 20 were then used: ML or LM sweep across the FOV in both hemispheres (FIG. 21F). The rectangular sweep both ML and LM sweeps significantly increased ipsiversive turning compared to controls, but there was no significant difference between different sweep directions (ML or LM) (FIG. 21G). If sweeping was conducted across the FOV over 5 seconds, both ML and LM sweeping stimulation produced more turning than controls, and LM produced significantly greater turning compared to ML sweeps (FIG. 21H).

### Methods

Experimental Subjects. All experimental procedures were approved by the Animal Care and Use Committee at Duke University. Male D1-Cre mice (Jackson Labs: Drd^{1tm2.1St1}) and A2A-Cre (Adora2A^{tm1Dyj/J}) mice were used. All mice were between 3-8 months old, group housed, and maintained on a 12:12 light cycle. Testing was always performed during the light phase.

Viruses. pAAV.CAG.Flex.NES-jRCaMP1b.WPRE.SV40 was a gift from Douglas Kim & GENIE Project (Addgene viral prep #100849; n2t.net/addgene:100849; RRID:Addgene 100849). pAAV-EF1a-double floxed-hChR2(H134R)-EYFP-WPRE-HGHpA was a gift from Karl Deisseroth (Addgene plasmid #20298; n2t.net/addgene:20298; RRID:Addgene_20298).

Surgery and Histology. Mice were initially anesthetized with 5.0% isoflurane and maintained at 1-2% during surgery. A craniotomy was made to allow implantation of the GRIN lens (Bregma+0.0-1.0 mm AP, ±2.0-2.7 ML). Pulled pipettes were used to inject the virus using a Nanoject III injector (Drummond Scientific, USA). The first virus injection (250 nl of pAAV.CAG.Flex.NES-jRCaMP1b.WPRE.SV40) was injected at two sites (+0.25, +0.75 AP, 2.5 ML) each with 5 depths (2.8-2.0 DV). Injections were made at a rate of 1 nl per second. The second injection (250 nl of AAV(9)-EFIa-DIO-hChR2(H134R)-EYFP) was then injected at the same coordinates. The injection pipette was always left in place for three minutes after each injection to allow for maximum absorption before it was retracted.

After the virus injection, aspiration was performed from brain surface, and a GRIN lens (1.8 mm×4.3 mm, Edmund Optics) was implanted in the DLS above the injection site. The lens was secured to the skull using dental cement and covered with Kwik-Sil to protect the lens surface. 5-6 weeks after the GRIN lens implantation, base plating was performed under visual guidance of the calcium signal to determine the best FOV.

After the completion of experiments, mice were transcardially perfused with 0.1M phosphate buffered saline (PBS) followed by 4% paraformaldehyde (PFA) to confirm placement and viral expression. The brains were then transferred to a 30% sucrose solution and sliced coronally using a cryostat (Leica CM1850). Slices were mounted with DAPI-mounting medium (Vector Laboratories, Vectashield, cat. no. H-1800) to identify the nuclei of neurons. Slices were then imaged using an inverted confocal microscope (Zeiss LSM780 and LSM880) for zoomed in images or an upright epifluorescence microscope for whole brain images (Axio Imager.M1—Zeiss).

Filters and LED Control. In this example, A lime LED and a blue laser were used. A LUXEON Rebel Color lime LED (LXML-PX02-0000) filtered with a 540-580 nm excitation LED filter (Chorma ET560/40x) was used for calcium imaging, while a 473 nm blue laser filtered with an excitation 450-490 nm laser filter (Chroma ET470/40x) was used for optogenetic stimulation. The two colors were combined through the excitation dichroic mirror (Chroma 59003bs) by merging them in the excitation light path. The main dichroic mirror (Chroma 69013bs) reflects the excitation light into the GRIN lens, while the fluorescence image passes through the emission filter. The emission filter (Chroma ET630/75) was designed to avoid crosstalk from the excitation light. In addition, an absorber (Chroma ET775/50x) was located beneath the DMD to avoid reflected blue light during the off-state that could potentially scatter into the excitation light path (FIG. 10).

Other suitable wavelengths for the single-photon light source and the laser can be selected. The wavelength for the single-photon light source is dependent upon the calcium indicator used in the subject.

The output from the lime LED was controlled by two parallel current chips (LT3092ETS8), both of which supply 2.9V of voltage and enough current to power the LED. The constant current source can provide up to 200 mA of current (up to 12 mW/mm² measured beneath the GRIN lens) with a step size of 4 mA. A programmable potentiometer (MCP4018) was used to adjust the current using control signals from an Arduino UNO (Arduino), which communicates with the computer. Custom scripts were used to adjust the parameters of calcium imaging.

Digital Micromirror Device (DMD) design and installation. For patterned stimulation, a digital micromirror device (DMD) was used as the spatial light modulator (Texas Instruments; DLP2010), which is a digitally controlled micro-opto-electromechanical system that modulates spatial light to generate different light stimulation patterns. This DMD has on its surface more than 400,000 microscopic mirrors arranged in a rectangular array, with a resolution ratio of 854×480 pixels. Each mirror has a hinge and hook beneath it, allowing it to rotate ±17° (relative to the flat surface), in order to switch between on- and off-states. The “on-state” is defined by each of the mirrors' positions corresponding to the tilt angle θ (±17°), such that the reflected light is directed toward the excitation light path. When a mirror is positioned in the opposite direction, the mirror is said to be in the “off-state”. In the on-state, light from the blue laser is reflected into the lens. In the off state, light is reflected into the absorber rather than into the GRIN lens, preventing stimulation in the specified FOV (FIG. 10). The rotation angle should be limited, so that in the off-state, all the light will be reflected to the absorber without any light scattering. Therefore, the DMD rotation angle must be limited as follows:

$$\{ \begin{matrix}2 ( α + θ ) < 90 ° \\ α + 3 θ < 90 °\end{matrix} \qquad ( 1 )$$

Where θ is the DMD rotation angle and α is the mirror tilt angle. α=13° was set as the optimal rotation angle.

Miniscope housing design and simulation in software. The miniscope body size is small, while the distance between the laser source and the GRIN lens is 20 mm. To generate precise patterns beneath the GRIN lens, and to have sufficient energy for excitation, the beam divergence for the laser light was than 4°. To generate a near perfect collimated light with sufficient energy in the small miniscope body, a laser fiber-head 100 μm in diameter was placed next to the focal point in order to gather all the energy and collimate the beam (FIG. 11). The collimation deviation was calculated by the following equation:

$$η = \frac{{R}_{D 1} - {R}_{D 50}}{{R}_{D 1}} \qquad ( 2 )$$

Where R_{D1} is the radius of the illumination field when the target screen is 1 mm from the outer surface of the collimation lens, R_{D50} is the radius of the illumination field when the target screen is 50 mm from the surface, and η is the collimation deviation. The smaller the deviation, the more precisely collimated the light is.

After the placement and operation of all the filters and lenses were confirmed, a 3D model housing all the components was designed in Solidworks software. To verify the optic simulation designs, all the 3D models were imported into the software program Tracepro (FIG. 10). The miniscope body was then printed using a 3D printer (Multi-Jet Fusion technology) with the material Nylon PA12.

Miniscope control system design. MAPSI includes: A CMOS imaging sensor, a PCB board, all the optical components such as the emission and excitation filters, the GRIN and achromatic lenses, and DAQ hardware and software. The image sensor resolution is 752px*480px and the frame rate is up to 60 Hz.

The whole system contains an LED power source, a recording subsystem and a pattern control subsystem (FIG. 12). A first computer was used that connects with all of the subsystems to send and receive data, as well as to send commands. A second computer was used to project the photo-stimulation patterns into the pattern control subsystem. The first computer runs two programs: the original miniscope recording software, and the integration software application that controls the LED power subsystem, which sends synchronous commands to all of the other subsystems to ensure timestamps are all aligned. An Arduino UNO that is connected with this computer sends and receives the synchronous commands from/to the CMOS to control the LED current and the pattern. A National Instruments (NI) box is also connected with this computer, and sends TTL signal to control the laser generator (RL639T8-500).

The DMD was controlled by a custom designed driver board with a 60 cm enameled wire cable. The DMD driver board was fixed on the following-up turning holder with the HDMI interface board. One computer sent image data to the HDMI interface using a wireless HDMI Kit (Diamond VS75). The pattern control subsystem receives the pattern image data from the computer, and controls the DMD (Texas Instruments; DLPC3430 and DLPA2000). An HDMI data interface board transmits signals from the computer to the DLP3430.

Determining z-axis resolution of MAPSI. The MAPSI z-axis resolution was determined in brain slices placed on a confocal microscope (Zeiss, LSM 880) equipped with a 20× (NA 0.8) objective. Using a vibratome, 200 μm coronal sections were cut from a mouse brain co-expressing RCaMP1b and ChR2. The slice was adhered to the bottom of a culture dish, and the GRIN lens was placed just above the slice. The GRIN lens and the tissue were then embedded together with 4% agarose gel. MAPSI was then fixed on the top of the GRIN lens, and attached to a stereotaxic frame to obtain a good focal pattern image (FIG. 13B). A confocal microscope was used to image the slice from bottom of the dish while simultaneously using MAPSI to target single neurons from the top of the dish. While MAPSI is generating a pattern on the tissue, RCaMP1b images were acquired using 561 nm excitation laser and 2 detectors (1 PMT for the pattern and 1 GaAsP detector for RCaMP) (pixel size, 0.21 μl×0.21 μl×0.70 μl; pinhole size, 1 airy unit). The total energy detected beneath the GRIN lens was measured using optical power meter (PM100A).

Calibration of MAPSI. To match DMD and CMOS pixel location, a 60 μm brain slice co-expressing jRCaMP1b and ChR2 was used. A GRIN lens was placed on the brain slice, and MAPSI was fixed on the top of GRIN lens. Once an image with white spot (diameter=10 pixels) and black background was projected on DMD, a spot fluorescence image was recorded on CMOS (FIG. 10G). As the white spot was moved in one axis, the movement of the fluorescence spot was also captured. Based on the DMD pixels (854*480) and the CMOS pixels (752*480), the white spot position and fluorescence spot position was calibrated with the linear relationship:

$$\frac{5 {P}_{DMD - X}}{7} + 166 = {P}_{CMOS - X} \qquad ( 3 )$$

Where P_{DMD-X} is the x axis position on DMD, and P_{CMOS-X} is the x axis pixel position on CMOS.

$$\frac{10 {P}_{DMD - Y}}{11} + 13 = {P}_{CMOS - Y} \qquad ( 4 )$$

Where P_{DMD-Y} is the y axis position on DMD, and P_{CMOS-Y} is they axis pixel position on CMOS.

Once a neuron is identified for stimulation, the pixels' location of that neuron can be imported into equation (3) and (4) to calculate the matching pixels' location on DMD. The projection image is then created with the calculated pixels.

Open-field behaviour recording and analysis. Mice were tested in an open field arena (25 cm×25 cm). A high-speed camera (FLIR BFS-U3-04S2M) was placed above the platform to record the behaviour of the mice at 50 frames/s. The DMD driver and HDMI interface board were fixed on the tuning holder, connected to the DMD using a 60c m enameled wire cable which prevents tangling of the wires and allows the mice to move freely. Other wires (coaxial cable for CMOS, power wire for LED) pass through the center hole of ball bearing (FIG. 12).

Behavioural data recorded by the cameras was analysed using DeepLabCut (Mathis, A. et al. DeepLabCut: markerless pose estimation of user-defined body parts with deep learning. Nature neuroscience 21, 1281-1289 (2018). After labeling several frames (skeleton with markers on the head and back of the mice), the behaviour data can be calculated by transfer learning with deep neural networks. In DeepLabCut, 3 markers (head, body, and tail) were labeled in each open-field behavioural movie. 200 samples of frames were auto selected from each video and 18 videos was taken for training with the 200,000 training iterations. The test error was 4.37 pixels and the training error was 1.63 pixels. The resulting data was then imported into MATLAB where behavioural variables, such as displacement and turning angle, were created using a custom script. The processed data was then imported into Neuroexplorer 5 along with the calcium imaging data for further analysis

Statistical Analyses. All statistical analyses were performed in GraphPad Prism 7.0. All error bars represent standard error of the means (SEM). Significance levels were set to P<0.05. Significance for comparisons: *P<0.05; **P<0.01; ***P<0.001; ****P<0.0001.

Calcium imaging acquisition and analysis. A CMOS imaging sensor, a data acquisition system (DAQ), and a USB host controller were used for calcium imaging. Images were acquired at 20 frames per second and recorded to uncompressed “.avi” files using the DAQ software. The videos were then imported into MATLAB for non-rigid motion correction, followed by deconvolution with the constrained non-negative matrix factorization (CNMF-E) algorithm. Seed pixels were initialized with a minimum local correlation value of 0.8 and a minimum peak-to-noise ratio of 10. The minimum number of nonzero pixels for each neuron was set at 10 based on the resolution of the CMOS sensor (1 μm/pixel). Both deconvolved and raw calcium traces were saved.

It is understood that the foregoing detailed description and accompanying examples are merely illustrative and are not to be taken as limitations upon the scope of the disclosure, which is defined solely by the appended claims and their equivalents. All publications and patents mentioned in the above specification are herein incorporated by reference as if expressly set forth herein. Various changes and modifications to the disclosed embodiments will be apparent to those skilled in the art and may be made without departing from the spirit and scope thereof.

1. A portable system for in-vivo imaging, comprising:
a) a single photon light source;
b) a dichroic mirror;
c) a freeform lens comprising an outer freeform surface and an inner plane;
d) an implanted lens placed in a tissue of a subject; and
e) an image sensor,
wherein light rays from the single photon light source refract through the outer freeform surface onto the dichroic mirror, and are reflected by the dichroic mirror onto the implanted lens, thereby transferring light energy from the single photon light source to an illumination area on the tissue of the subject; and
wherein the light energy transferred from the single photon light source to the tissue of the subject illuminates a detectable signal from a calcium indicator present in the tissue of the subject, and an image of the detectable signal is captured by the image sensor.

2. (canceled)

3. The portable system claim 1, wherein the outer freeform surface is designed such that light rays refracted through the outer freeform surface contact the dichroic mirror in locations that achieve a substantially even distribution of light rays relative to a center point within the illumination area following reflection.

4. (canceled)

5. (canceled)

6. The portable system of claim 1, wherein the image sensor is a complementary metal oxide semiconductor (CMOS) image sensor and/or wherein the implanted lens is a gradient index (GRIN) lens.

7. (canceled)

8. (canceled)

9. The portable system of claim 1, wherein the maximum distance between a point at the center of the inner plane and a point on the outer freeform surface is 1.5 mm or less, the illumination area is a substantially circular area having an average diameter of at least 150 microns, and/or the system weights 5 g or less.

10. (canceled)

11. (canceled)

12. (canceled)

13. (canceled)

14. (canceled)

15. (canceled)

16. A portable system for in-vivo two-color calcium imaging, comprising:
a) a first light source and a second light source;
b) an excitation dichroic mirror;
c) an excitation lens;
d) a main dichroic mirror;
e) an implanted lens placed in a tissue of a subject; and
f) a first image sensor and a second image sensor,
wherein light rays from the first light source and the second light source are integrated into a main excitation path by reflection from the excitation dichroic mirror onto the excitation lens, are subsequently refracted through the excitation lens onto the main dichroic mirror, and are then reflected by the main dichroic mirror onto the implanted lens, thereby transferring light energy from the first light source and the second light source to an illumination area on the tissue of the subject, and
wherein the excitation dichroic mirror is positioned such that the illumination area contains a substantial majority of the light rays from each of the first light source and the second light source.

17. (canceled)

18. The portable system of claim 16, wherein the first light source and the second light source are single photon light sources, and/or wherein the portable system further comprises a first light filter operably connected to the first light source to select for light of a first wavelength, and a second light filter operably connected to the second light source to select for light of a second wavelength.

19. (canceled)

20. The portable system of claim 16, wherein the light energy transferred to the tissue from the first light source illuminates a first detectable signal from a first calcium indicator present in the tissue of the subject, and wherein the light energy transferred to the tissue from the second light source illuminates a second detectable signal from a second calcium indicator present in the tissue of the subject.

21. The portable system of claim 20, wherein the portable system further comprises an achromatic lens and an emission dichroic mirror, wherein the first detectable signal and the second detectable signal each refract through the achromatic lens onto the emission dichroic mirror, and are subsequently split by the emission dichroic mirror, thereby reflecting the first detectable signal to the first image sensor and the second detectable signal to the second image sensor.

22. The portable system of claim 16, wherein the implanted lens is a gradient index (GRIN) lens, the first image sensor and the second image sensor are each complementary metal oxide semiconductor (CMOS) image sensors, and/or the system weight 5 g or less.

23. (canceled)

24. (canceled)

25. (canceled)

26. (canceled)

27. The portable system of claim 16, wherein the illumination area is a substantially circular area having an average diameter of at least 150 microns.

28. (canceled)

29. (canceled)

30. A portable system for combined modulation and imaging of cellular activity in vivo, the portable system comprising:
a) a laser;
b) a collimation lens;
c) a spatial light modulator (SLM);
d) a single photon light source;
e) an excitation dichroic mirror;
f) an excitation lens;
g) a main dichroic mirror,
h) an implanted lens placed in a tissue of the subject; and
i) an image sensor.

31. The portable system of claim 30, wherein beams from the laser refract through the collimation lens to generate collimated laser beams, wherein the collimated laser beams are reflected by the spatial light modulator to generate a patterned excitation light path which is reflected by the main dichroic mirror onto the implanted lens, thereby transferring the patterned excitation light path to a stimulation area on the tissue of the subject and inducing patterned modulation of cellular activity within the stimulation area, and
wherein light rays from the single photon light source are reflected by the excitation dichroic mirror onto the excitation lens, refract through the excitation lens onto the main dichroic mirror, and are subsequently reflected by the main dichroic mirror onto the implanted lens, thereby transferring light energy from the single photon light source to an illumination area on the tissue of the subject,
wherein the illumination area and the stimulation area are substantially the same region within the tissue.

32. (canceled)

33. (canceled)

34. (canceled)

35. (canceled)

36. (canceled)

37. The method of claim 30, wherein the illumination area and the stimulation area are each substantially circular areas having an average diameter of at least 150 microns.

38. (canceled)

39. (canceled)

40. The portable system of claim 30, wherein the light energy transferred from the single photon light source to the tissue of the subject illuminates a detectable signal from a calcium indicator present in the tissue of the subject, and wherein an image of the detectable signal is generated by the image sensor.

41. The portable system of claim 30, further comprising an excitation filter operably connected to the single photon light source to select for light of a first wavelength.

42. (canceled)

43. (canceled)

44. The portable system of claim 30, wherein the image sensor is a complementary metal oxide semiconductor (CMOS) image sensor, wherein the implanted lens is a gradient index (GRIN) lens, wherein the excitation lens is an achromatic lens, and/or wherein the portable system weights 8 g or less.

45. (canceled)

46. (canceled)

47. (canceled)

48. (canceled)

49. A method for combined manipulation and imaging of cellular activity in vivo, the method comprising:
a. connecting the portable system of claim 30 to a tissue of the subject;
b. generating beams from the laser, thereby transferring a patterned excitation light path to a stimulation area on the tissue and inducing a patterned manipulation of cellular activity for one or more cells within the stimulation area;
c. generating light rays from the one-photon light source, thereby illuminating a detectable signal from an indicator present in the tissue of the subject; and
d. obtaining an image of the detectable signal.

50. The method of claim 49, further comprising converting the image of the detectable signal to a readout of a cellular activity pattern of one or more cells in the subject.

51. The method of claim 50, further comprising mimicking the cellular activity pattern of the one or more cells, wherein mimicking the cellular activity pattern of the one or more cells comprises selectively targeting the one or more cells with the patterned excitation light path, such that a patterned modulation of activity for the one or more cells is induced.

52. (canceled)

53. The method of claim 49, wherein the indicator is a calcium indicator and/or wherein the one or more cells comprise neurons.

54. (canceled)
